(19) United States
(12) Patent Application Publication
(10) Pub. No.: US 2003/0036207 A1
(43) Pub. Date: Feb. 20, 2003

(54) System and method for storing mass spectrometry data

(76) Inventors: Michael P. Washburn, Encinitas, CA (US); Cosmin Deciu, San Diego, CA (US); Antonius A. Koller, Encinitas, CA (US); Ryan R. Ulaszek, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/195,845

(22) Filed: Jul. 12, 2002

Related U.S. Application Data
(60) Provisional application No. 60/305,289, filed on Jul. 13, 2001.

(51) Int. Cl. G01N 33/543; G06F 19/00; G01N 33/48; G01N 33/50

(52) U.S. Cl. 436/518; 702/19

(57) ABSTRACT

The present invention relates to a system and methods for facilitating the analysis of proteomic expression data. In this system, complex sequence-correlated peptide expression information and mass spectrum data are processed and stored in a relational database. Using a parallel computational method, the expression data and results are parsed and associated to rapidly yield peptide sequence information. The system automates necessary tasks associated with peptide data analysis and organizes large amounts of information needed to perform the data analysis in a logical and accessible manner.

### Related Applications

This application claims priority from U.S. Provisional Application No. 60/305,289, filed on Jul. 13, 2001.

### Field of the Invention

This invention relates to systems and methods for automatically calculating information received from a mass spectrometer. More specifically, this invention relates to systems and methods that process and store mass spectrometry data and information in a proteomic database.

### Background of the Invention

Recently, considerable effort has been made to develop mass spectrometric techniques and computational methods which correlate peptide and peptide mass spectral data with computer databases in order identify peptides (Eng et al., J Am Soc Mass Spectrom 5:976-980 (1994); Mann and Wilm, Anal Chem 66:4390-4399 (1994); Yates et al., Anal Chem 67:1426-1436 (1995)). These mass spectrometry based techniques for peptide identification identify peptide fragments based on a spectral signature uniquely generated for each peptide sequence. In the mass analysis procedure, a peptide mixture is separated using a first mass spectrometer which separates the peptides according to their mass and charge characteristics to produce a spectrum indicative of the component peptides of the peptide mixture. Each separated peptide is then further subjected to a second tandem mass analysis where the peptide is fragmented and a second mass spectrum is produced. The second mass spectrum comprises a series of peaks (peptide signature) formed as a result of differences in the mass-to-charge ratios of fragments of the peptide. For peptides with differing sequences, the series of peaks uniquely identifies the particular sequence of the peptide undergoing analysis.

Computational methods for sequencing peptides subjected to mass analysis involve comparing the spectrum generated by the peptide of interest with known spectra. In these methods, the peptide spectrum is associated with a known sequence to indicate sequence homology. The results of the analysis typically contain many values and statistical correlations that, when taken together, identify associations between the peptide signature and the known spectra. The analysis may also include candidate sequences that are likely to match the experimental spectrum, as well as, correlation scores and probabilities indicating the degree of confidence of the match. These results are typically output to the investigator in the form of complex tables or output files which present large amounts of data and information which must be carefully reviewed in order to accurately assess the peptide analysis.

U.S. Pat. No. 6,017,693 describes a system for correlating a peptide fragment mass spectrum with amino acid sequences derived from a database. This is one example of a conventional mass spectrometry-based method for peptide identification which compares an experimental peptide spectrum with a known database of spectra. In this system, mass spectra from an experiment are input into a computer containing a database of sequence-associated spectrum. The computer then performs a search of the database and outputs results of the search to the investigator in the form of an output file or summary.

A drawback encountered when using the aforementioned peptide sequencing system results from the production of complex output files which may obfuscate and slow down the speed with which interpretation of the data and information can be performed. At least a portion of this problem arises as a result of the requirement for the data and information to be reviewed and interpreted manually by the investigator to determine or confirm the peptide sequence. As a result, such a system may be employed to process a relatively small sample peptide population, however, its utility is severely diminished when assessing the many thousands of proteins or peptides typically present in a cell or tissue extract. The resulting amount of time an investigator must devote to reviewing the output files of the analysis therefore represents a significant bottleneck which must be alleviated if complex mixed-populations of peptides are to be assessed.

Thus, in the analysis of complex mixed peptide samples, there is a need for an automated method for processing mass spectral data in which peptide signatures generated during an experiment can be automatically queried against a database of spectral information to generate sequence information. Additionally, there is a need for a system which receives the results from the peptide sequence analysis, processes the results and performs an interpretation automatically. Such a system is useful when identifying and comparing large numbers of proteins or peptides as are typically found in whole cell or tissue extracts. Furthermore, this system should be adapted to store the information in a central database permitting the comparison of results obtained from many experiments to facilitate global proteomic comparisons and data mining operations.

### SUMMARY OF THE INVENTION

Embodiments of the invention include a mass spectrometry-based system and method for rapidly and quantitatively analyzing peptides in complex mixtures or isolates. The system also features automated processing capabilities used to analyze differentially expressed peptides in a single sample in order to reduce variability and increase accuracy. Differentially expressed peptides are identified by changes in expression patterns which, for example, may be affected by a stimulus (e.g., administration of a drug or contact with a potentially toxic material), by a change in environment (e.g., nutrient level, temperature, passage of time) or by a change in condition or cell state (e.g., disease state, malignancy, site-directed mutation, gene knockouts) of the cell, tissue or organism from which the sample originated.

### BRIEF DESCRIPTION OF THE DRAWINGS

These and other aspects, advantages, and novel features of the invention will become apparent upon reading the following detailed description and upon reference to the accompanying drawings. In the drawings, same elements have the same reference numerals in which:

FIG. 1 is a flow diagram illustrating a differential peptide identification methodology.

FIG. 2 is a block diagram illustrating a data analysis system used to identify differential peptide expression.

FIG. 3 is a flowchart illustrating a method of qualitative analysis of complex peptide mixtures.

FIG. 4 is a simplified mass spectrum intensity curve for a differentially labeled peptide in which markers create a mass differential between analogous peptides.

FIG. 5 is a flowchart illustrating a correlation process used for identifying differentially labeled peptides.

FIGS. 6A-E are simplified mass spectrum scans illustrating states of differential expression that may be identified by the data analysis system.

FIG. 7 is a flow diagram illustrating a method for identifying and quantitating chromatographic peaks from a differentially labeled mass spectrum analysis.

FIG. 8 is a flow diagram illustrating a method for parallel processing of mass spectrum and sequence data.

FIG. 9 is a flow diagram illustrating computational activities performed by nodes within a parallel architecture that are used to resolve and quantitate differentially expressed peptides.

### DETAILED DESCRIPTION OF THE PREFERRED EMBODIMENT

The system and methods presented herein are useful in identifying protein or peptide components when comparing mixed peptide populations for differential expression. In one embodiment, each population is labeled with an identifiable label or marker to resolve the mixed-population of peptides within the same sample or analysis. The resulting combined analysis provides improved resolution and identification capabilities and is not subject to the degree of instrumental or cross-sample experimental variations which confound conventional peptide identification techniques.

The peptide identification system further implements an automated sequencing routine in which tandem mass spectra identification resolves protein sequences by querying and correlation against a spectral database of known peptide spectra. This feature significantly improves data acquisition and sequencing throughput and provides a mechanism by which peptides within the mixed-population can be readily identified without additional sequencing steps or reactions.

As described below, in one embodiment an affinity labeling procedure is used to selectively isolate peptides that contain a desired label or tag. The isolated proteins, peptides, or reaction products are then characterized by mass spectrometry (MS) based techniques. In particular, the sequence of isolated peptides is determined using tandem MS (MS)ⁿ techniques which are correlated with known peptide spectrum produced by the tandem MS (MS)ⁿ techniques.

Prior to spectrometric analysis, the system for peptide identification and differential comparison incorporates a chromatographic/separation technique, such as microcapillary liquid chromatography or gas chromatography. These chromatographic techniques separate the mixed peptide sample or solution of interest thereby permitting selective analysis of each peptide sequence. Following the preliminary separation of the components, the sample is introduced into a mass spectrometer which serves as a detector of the individual components. Such a coupling between of these two technologies provides an efficient and high resolution method to identify the individual peptide components contained in the sample of interest.

The spectral database comprises a collection of tandem mass spectra which have been previously associated with known peptide sequences. One example of a mass spectral database is described in U.S. Pat. No. 5,538,897 to Yates, et al. Software comparison and identification routines correlate the output spectrum from mass spectrometry of the sample with those spectrum contained in the spectral database and returns the peptide identity of each peptide in the sample. Using these methods the spectrum of a complex peptide mixture is readily resolved and the corresponding sequences of the constituent peptides are identified as will be described in greater detail hereinbelow.

The following discussion provides examples of differential comparisons that are made based on treated and untreated cell or tissue populations. However, it will be appreciated that the peptide identification methods presented herein provide a flexible means for conducting comparisons between many different types of samples. Thus, these methods are applicable to a variety of instances where it is desirable to study differential peptide expression between two or more peptide populations. For example, in addition to comparing a treated versus untreated cell or tissue population, comparisons between different cell or tissue types may also be made. Furthermore, the analytical methods described herein can be used for multiplex analysis to simultaneously assess a complex mixture of peptides derived from more than two samples or peptide populations.

A. System Overview

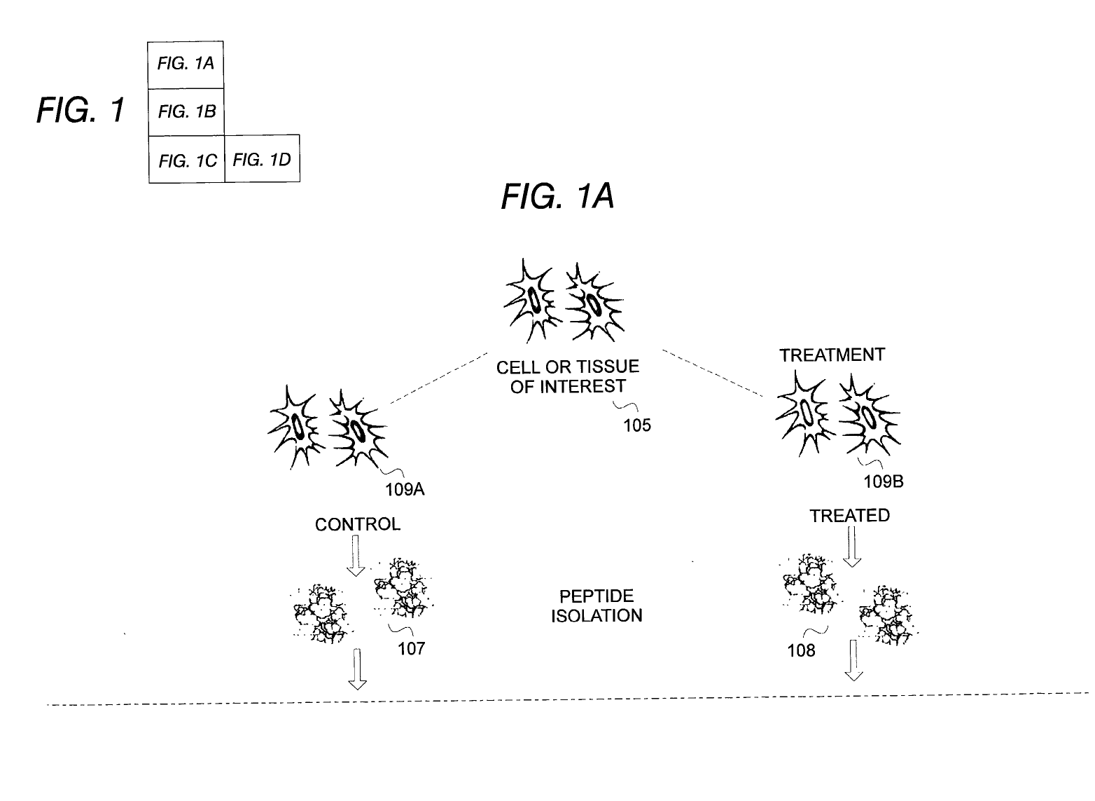

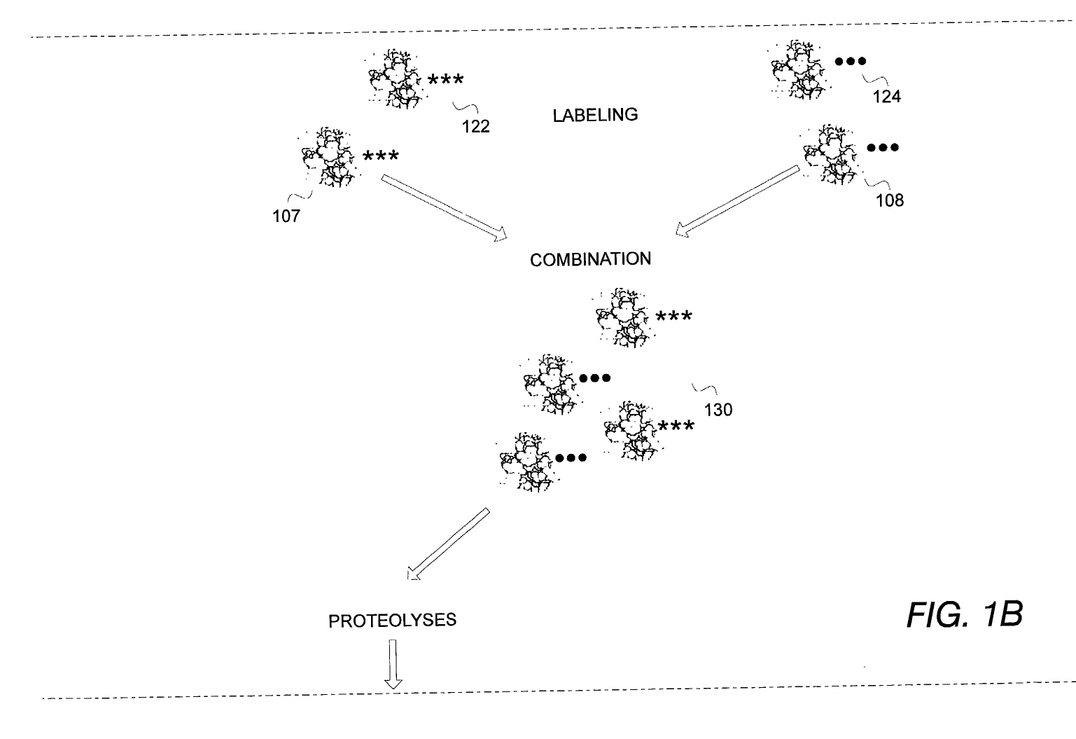

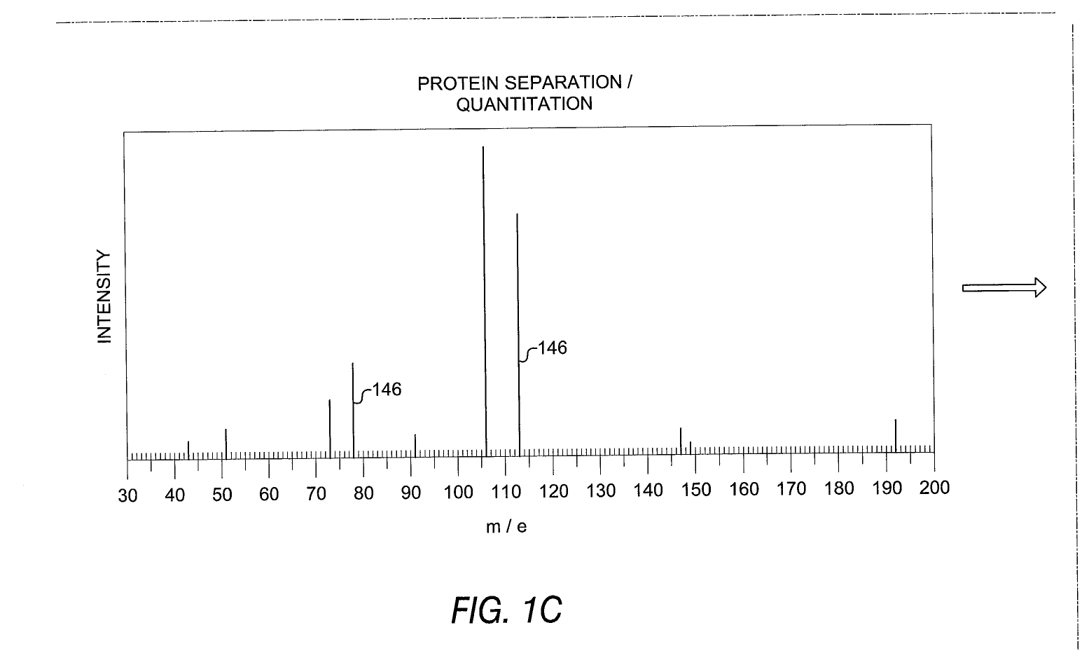

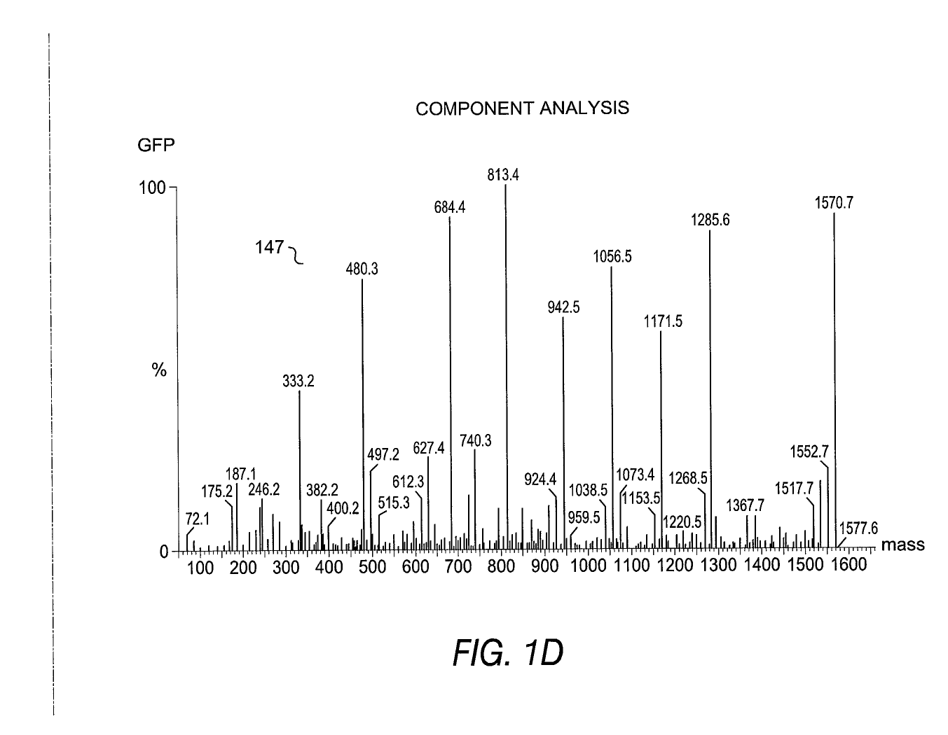

FIG. 1 illustrates an overview of one embodiment of a peptide identification and differential analysis technique used to resolve, sequence, and identify complex peptide mixtures derived from two or more peptide populations. A typical comparison of differential expression is made using a starting cell population 105. One portion of the cell population 105 is separated into a control cell population 109A, while another portion of the population 105 is treated with a test compound to become test cell population 109B.

The test cell population 109B is treated with one or more conditions or treatments for which proteomic differences are to be identified. In one exemplary embodiment, the cell population 105 is analyzed by comparing the proteomes of the control population 109A with the treated cell population 109B.

Once the cells have been treated, the protein or peptide populations from each cell are isolated to yield a control peptide population 107 and a treated peptide population 108. During this stage of analysis the peptide isolation procedure may additionally incorporate processing or purification steps designed to remove undesirable or contaminating biomolecules and chemicals. For example, during the harvest of peptides from a cell or tissue, biomolecules such as RNA, DNA, and proteases, as well as, extraction reagents and buffers may be removed from the peptide isolate to prevent interference with detection of the peptide molecules.

A subsequent labeling reaction is used to label each peptide population 107, 108 with an identifiable peptide labeling moiety or label 122, 124 which aids in resolving the peptide populations 107 during mass analysis. In one aspect, the labels 122, 124 comprise multi-functional synthetic peptide sequences with differing masses. During the analysis, the peptide populations 107, 108 are made differentially identifiable by incorporating the first label 122 into the first peptide population 107 and incorporating the second label 124 into the second peptide population 108. Thus, the peptides 107, 108 derived from each condition or treatment 110 are made to contain an identifiable label 122, 124 of known mass. The difference in molecular weight between the first label 122 and the second label 124 serves as a basis for determining the peptide population 107, 108 of origin from which an identified peptide is derived by creating a mass differential between the two peptide populations. Examples of differential labels are described below.

The labels 122, 124 may additionally contain a peptide epitope tag or motif used for affinity purification of the labeled peptides 107, 108. This feature of the labels 122, 124 is useful for isolating only those peptides which have been labeled and may further serve as a means for enriching the peptide populations 107, 108. Enrichment of the peptide populations 107, 108 increases the sensitivity of the mass detection procedure and removes background “noise” that may be contributed by unlabeled or undesirable peptides.

Of course, it is not required to label both populations of peptides. Accordingly, only the treated peptide population 108 might be labeled in order for each peptide in the treated population to have a different mass from the control population. Additionally, it is contemplated that the peptides can be metabolically labeled prior to isolation from the cells or tissues. In this alternative method, discernable peptide populations 107, 108 are created through the use of isotopic labeling to create peptide populations 107, 108 with differing masses. In metabolic labeling, a heavy isotope label, such as a nitrogen isotope (¹⁵N), may be incorporated into the first peptide population 107 and a lighter nitrogen isotope, such as ¹⁴N, may be incorporated into the second peptide population 108. The different isotopes are incorporated in-vivo to label all of the amino acids to create the discernable peptide populations without the requirement of a subsequent labeling step.

When using the peptide epitope tag for affinity purification, a specific protease site may further be incorporated into the label 122, 124 to facilitate the release of the affinity purified labeled peptides from an affinity matrix. Additional details of the chemical composition of the labels 122, 124 as well as details of the specialized peptide epitope motifs for purification of the peptide populations 107, 108 are described below.

Following peptide labeling, cleanup and purification procedures may be used to prepare the peptide populations 107, 108 for analysis. The control and treated peptide populations are then combined to form a single mixed-population peptide sample 130. Combining the uniquely labeled peptide populations 107, 108 in this manner desirably simplifies subsequent mass analysis procedures while permitting peptides from each population 107, 108 to be resolved, identified, and compared using the incorporated labels 122, 124.

Furthermore run-to-run inconsistencies, experimental variabilities, and user-induced inaccuracies are minimized by combining the peptide samples 107, 108 to result in improved data output and more definitive peptide identification. The improvement in analysis is due, in part, to the observation that by the combining peptide samples, the two peptide populations 107, 108 are subjected to identical conditions and manipulations thus reducing variability between the samples which would otherwise be treated and analyzed independently.

In preparation for mass analysis, the mixed peptide sample 130 is subjected to proteolysis to fragment the peptides 107, 108 into smaller molecules which are of suitable size for use in mass spectrometry-based techniques. Furthermore, protease cleavage can be used to release labeled peptides 107, 108 from the aforementioned affinity matrix.

Proteolysis is desirably conducted using a highly specific protease enzyme. Examples of protease enzymes which may be used for peptide digestion include: TEB protease, chymotrypsin, endopeptidease Arg-C, endopeptidease Asp-N, trypsin, Staphylococcus aureus protease, thermolysin, and pepsin. As described in greater detail below, protease selection may be directed by the type of label incorporated into the labeled peptides 107, 108. These labels 122, 124 may contain amino acid sequences which define specific protease cleavage sites which are designed to release the labeled peptides from the affinity matrix to provide a purified or enriched peptide sample.

Quantitation of peptide expression levels is performed using mass analysis techniques which determine peptide quantities within the differentially labeled mixed-population peptide sample 130. As discussed above, in one embodiment, the mixed-population sample 130 is first subjected to a preliminary separation step using liquid or gas chromatography methods or 2-dimensional gel electrophoresis. In another embodiment multidimensional protein identification technology (MudPIT) (Washburn et al., Nature Biotechnology, 19: 242-247 (2001)) is used as a preliminary means to separate the peptide components resulting from the aforementioned proteolysis reactions.

The MudPIT technique utilizes a fused-silica microcapillary column packed with a reverse-phase material (XDB-C18, Hewlett-Packard, CA) in addition to a strong cation exchange material (Partisphere SCX, Whatman, NJ). The mixed-peptide sample is loaded onto the packed column and placed in-line with the mass spectrometer and a buffer solution is passed through the column to elute the peptides. The resulting peptide eluate provides a preliminary separation means for the peptides which are then passed through the mass spectrometer resulting in further separation of the peptides according to their mass-to-charge ratio.

As will be appreciated by one of skill in the art, numerous methodologies exist which may be used to provide a preliminary separation means for resolving the mixed-peptide sample prior to mass analysis. Thus, these preliminary separation means used in conjunction with the mass analysis techniques described herein represent alternate embodiments of the present invention.

The mass spectrometer, in addition to serving as a peptide-separation means, acts as a detector to provide information useful in the identification of each peptide species contained within the mixed-population sample 130. Mass analysis, in this manner, provides a suitable method to compare expression levels between similar peptides 107, 108 derived from different sources, conditions, or treatments as will be described in greater detail hereinbelow.

As will be appreciated by one of skill in the art, a number of mass analysis techniques may be applied to the resolution and identification of the mixed-population peptide sample 130. Examples of suitable mass analysis techniques include: electron ionization, fast atom/ion bombardment, matrix-assisted laser desorption/ionization (MALDI), and electrospray ionization. MALDI spectroscopy techniques in particular possess a number of desirable characteristics which improve the quality of the mass analysis. These characteristics include: large mass range of the input peptide species (greater the 300,000 daltons), high sensitivity (low picomole detectability), soft ionization (producing little or no observed fragmentation of the peptides), salt tolerance (in millimolar concentrations), and the ability to analyze complex mixtures of peptides in a resolvable manner.

Following the initial separation/quantitation step, a subsequent component analysis step is performed in which resolved peptides 146 undergo tandem mass analysis (MS(MS)ⁿ) to produce a unique spectrum 147 characteristic of the particular sequence of the peptide 146. In one embodiment, MS (MS)ⁿ spectrum 147 are desirably acquired for each resolved peptide 146 using an automated procedure wherein the individual spectrum 147 are acquired and stored for later processing and sequence identification.

In a typical differential expression and characterization analysis, a large number of MS(MS)ⁿ spectrum 147 are generated (at least one for each resolved peptide 146). While it is possible to visualize, review, and identify each spectrum manually, it is impractical and time consuming for an entire peptide population to be analyzed in this manner. Instead the MS(MS)ⁿ spectrum 147 are well suited to be processed by an automated method using computer assisted identification in conjunction with a spectral or correlative database, as will be described in greater detail hereinbelow.

Based on the aforementioned overview, differential peptide analysis compares peptides present in two or more biological samples. The peptides are labeled with a discernable marker to allow the peptides from each biological sample to be identifiable from one another when they are combined. Combination of the samples is desirable as it permits simultaneous analysis of the peptides and provides a means of directly comparing related peptides. Direct peptide comparison is further useful in identifying expression differences between related peptides within the two or more biological samples and aids in the detection of novel peptides.

For example, in a peptide population A and a peptide population B derived from a similar cell or tissue type, it will be expected that the composition of the two peptide populations will be related (i.e. both cells will contain identical peptides which may be expressed at different levels). The differential peptide analysis identifies and quantitates the relative concentrations of the related peptides in these populations to provide information about the overall peptide expression state of each biological sample. This analysis further identifies differences in peptide expression between the two biological samples which are useful in determining the effect of a treatment or condition upon a cell or tissue.

Peptides are identified using mass analytical methods in which the peptides undergoing analysis are bombarded with an electron beam to produce identifiable fragments (cations and radical cations) that are accelerated in a vacuum through a magnetic field and are sorted on the basis of mass-to-charge ratios. Peptides are identified on the basis of the mass-to-charge ratio which is related to the molecular weight of the fragments produced. Subsequent tandem mass analysis produces a unique spectral signature for each identified fragment which is compared to a database of known spectral signatures and used to identify the sequences of the collection of peptide fragments. One device for performing this function is a tandem mass spectrometer LCQ Deca from Thermo Finnigan (San Jose, Calif.). See http://www.thermofinnigan.com on the Internet for more information.

This embodiment of the invention therefore is an automated method for identifying the many thousands of component peptides (i.e.: the proteome) of a biological sample. Furthermore, the expression levels of the component peptides can be rapidly quantitated and compared between samples to give a better understanding of global peptide expression within biological systems.

B. The Data Analysis System

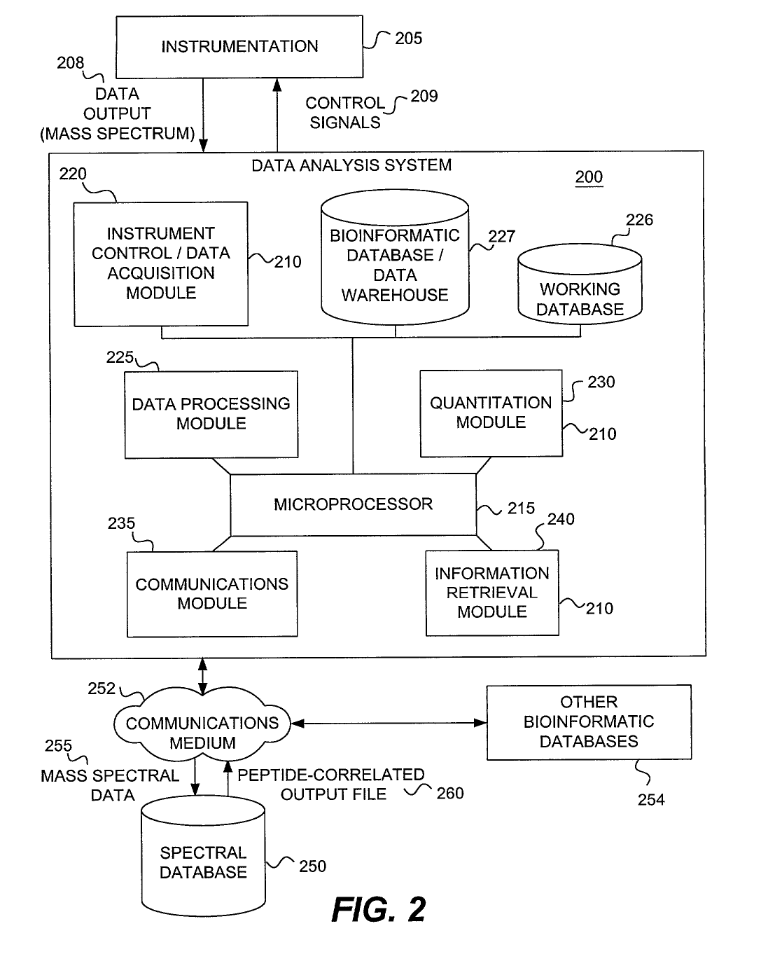

FIG. 2 illustrates components of a data analysis system 200 which interact with instrumentation 205 used to perform the differential peptide analysis. The data analysis system 200 comprises a plurality of modules 210 that operate in conjunction with a microprocessor 215 to receive and process data output 208 produced by the mass analysis and MS (MS)ⁿ techniques. Using these modules 210, the data analysis system 200 identifies the peptide constituents whose mass spectrum and associated information make up the data output 208 and subsequently processes the data to obtain detailed sequence and expression information.

In the illustrated embodiment, an instrument control/data acquisition (ICDA) module 220 acts as an interface between the instrumentation 205 and the data analysis system 200. The ICDA module 220 receives the data output 208 and performs necessary handshaking and error correcting functions to insure data integrity. The ICDA module 220 is further equipped to recognize and process various data types associated with the data output 208 which are native to the instrumentation being used 205. The ICDA module 220 may additionally issue control signals 209 which coordinate run-time activities associated with the instrumentation 205. For example, the control signals 209 may be used to modify configuration settings or parameters the instrumentation 205, as well as, manage operational modes such as starting/stopping sample analysis. Furthermore, control signals 209 may be issued by the data analysis system 200 to direct a plurality of mass spectral analysis scans to be acquired by the instrumentation 205 over a specified time period or with a particular frequency. In this embodiment, the mixed-peptide population 130 is eluted from the preliminary separation means and passed through the mass analysis instrumentation over a time period of approximately 1-10 minutes. During this time, mass spectral scans are taken with a frequency of approximately 50 scans/sec generating a plurality of mass spectral scans which are representative of the peptide composition at various points throughout the peptide elution. As will be described in greater detail hereinbelow, this method of multiscan mass analysis is used to construct peptide elution profiles for each of the peptides in the mixed population and improves the ability of the data analysis system 200 to identify and quantify proteomic differences.

A data processing (DP) module 225 receives the data output 208 from the instruments 205, formats the data output 208, and stores it in a working database 226 in a suitable form for later retrieval and processing. Functions of the DP module 225 may include rearranging or organizing the data output 208, performing operations to transform or change the format of the data output 208, or other tasks to prepare the data output 208 for subsequent analysis. The DP module 225 additionally interacts with a working database 226 (used to store raw data and information) and a bioinformatic database or data warehouse 227 (used to archive the experimental results after the data has been processed and the mixed-peptide population analyzed, quantitated, and compared) to organize, categorize and store the data output 208 in a form that may be easily sorted, queried, and retrieved.

The working database 226 and the bioinformatic database 227 are desirably implemented using relational schemas to provide flexible analytical querying and data mining capabilities. Furthermore, use of the databases 226, 227 provide a means by which the data output 208 and expression results may be correlated with other information creating an integrated bioinformatic system. In one embodiment, the databases 226, 227 may be implemented using applications designed for relational database development and implementation, such as those sold by Oracle Corporation (Redwood Shores, Calif.), Sybase Corporation (Emeryville, Calif.), and MySQL AB (Postgirot, Stockholm, Sweden). In other embodiments, the databases 226, 227 comprise database designs implemented using numerous other programming languages such as JAVA, C/C++, Basic, Fortran, or the like, wherein the database structure, tables, and associations are defined by code of the programming languages.

It is also recognized that other types of databases may be used, such as object oriented databases, flat file databases, and so forth. Furthermore, the databases 226, 227 may be implemented as a single database with separate tables or as other data structures that are well known in the art such as linked lists, binary trees, and so forth. Additionally, the databases 226, 227 may be implemented as a plurality of databases which are collectively administered to store and analyze the data of the data analysis system 200.

As will be subsequently described in greater detail, a communications module 235 of the data analysis system 200 interacts with a spectral database 250 to aid in the determination of the origin and sequence for each peptide component of the mixed peptide population under study. The spectral database 250 comprises stored spectra of known peptide sequences used to identify peptides from experimental tandem mass spectrum data 255. The data analysis system 200 desirably utilizes a computer program or search routine to identify the peptides by comparison of tandem mass spectrum data 255 with the spectral database 255. One such program for determining the identity of a peptide by matching tandem mass spectrum data with stored peptide spectra is the SEQUEST peptide identification program developed at the University of Washington (http://www.washington.edu). Information on the SEQUEST program and system can be found on the Internet at http://thompson.mbt.washington.edu.

Once the system 200 has searched the spectral database 250 in order to match tandem mass spec data with stored spectral data 208, peptide-correlated output files 260 containing the putative identities of the peptides determined from the spectral data analysis are then returned to the data analysis system 200 for further processing.

In one embodiment, communication between the data analysis system 200 and the spectral database 250 occurs by way of a communications medium 252, such as the Internet, with the communications module 235 providing functionality for sending and receiving data through a suitable means, such as a TCP/IP based protocol. The communications module may additionally provide accessibility to other remotely located bioinformatic information systems 254 such as GenBank, SwissProt, Entrez, PubMed, and the like to acquire other information which may be associated with the peptide-correlated output files 260 and information stored in the databases 226, 227.

A quantitation module 230 is used by the data analysis system 200 to determine more precise relationships between the peptides identified in the mixed-population and their relative expression levels. This module confirms the identity of each peptide in the mixed population of peptides by evaluating the results of the peptide correlated output files 260 and the mass spectrum data 208.

More specifically, the quantitation module 230 evaluates the peptide-correlated output files 260 and identifies peaks or intensity curves corresponding to resolved peptides in the mass spectrum data 208. The quantitation module 230 also quantitates the amount of peptide associated with a particular resolved peak 146 or intensity curve within the mass spectrum data 208 by area calculations. Additionally, the quantitation module 230 identifies and evaluates the peaks corresponding to the same peptide from both control and treated samples. This process will be described in greater detail hereinbelow.

As previously indicated, peptides from the control population and the treated population may be determined by the differential masses of the labels 122, 124 which are integrated into each peptide undergoing analysis. The use of the label 122, 124 distinguishes analogous peptides from different samples which have similar spectrum 208 by creating a mass differential between the analogous peptides containing different labels 122, 124. Identification of the peptides derived from each treatment or condition provides a means for the quantitation module 230 to perform cross-sample comparisons and identify changes in peptide expression.

The IR module 240 provides additional insight into the mixed population peptide samples under study by retrieving information from other bioinformatic databases 254 that may be correlated with peptide sequences identified by the data analysis system 200. For example, the IR module 240 may read information stored in the working database 226 or the bioinformatic database 227 and perform automated information search queries directed towards collecting additional information about the identified peptides. The IR module 240, therefore, provides an additional means for automatically associating bioinformatic information from other informational sources and repositories with the experimentally identified peptides to yield a detailed collection of information.

Based on the aforementioned system architecture, peptide expression data is acquired for the mixed population of differentially labeled peptides 130 and subsequently processed to identify the peptide constituents of the mixed population sample. The system 200 formats and stores the data in an organized manner and extracts relevant information to use to query the spectral database 250. The spectral database 250 then returns correlated tandem mass spectra 260 which are associated with the spectra of individual peptides in the mixed population undergoing analysis.

Typically, many thousands of queries are generated by the system 200 and the amount of information returned from the spectral database 250 necessitates an automated method for identifying and quantitating the peptide constituents of the mixed population 130. To this end, specialized modules 210 of the system 200 provide instructions which parse and process the correlated tandem mass spectra 260 in a rapid and efficient manner and store the results of the analysis in the bioinformatic database 227 for subsequent evaluation by the investigator.

As will be appreciated by one of skill in the art, the aforementioned automated analysis and correlation features of the data analysis system 200 free investigators from having to perform lengthy searches and associations on an individual basis. Furthermore, the data analysis system 200 provides a more complete collection of data and information to which subsequent data mining techniques can be applied to further investigate the components of the mixed-peptide population.

C. Analyzing Complex Mixtures

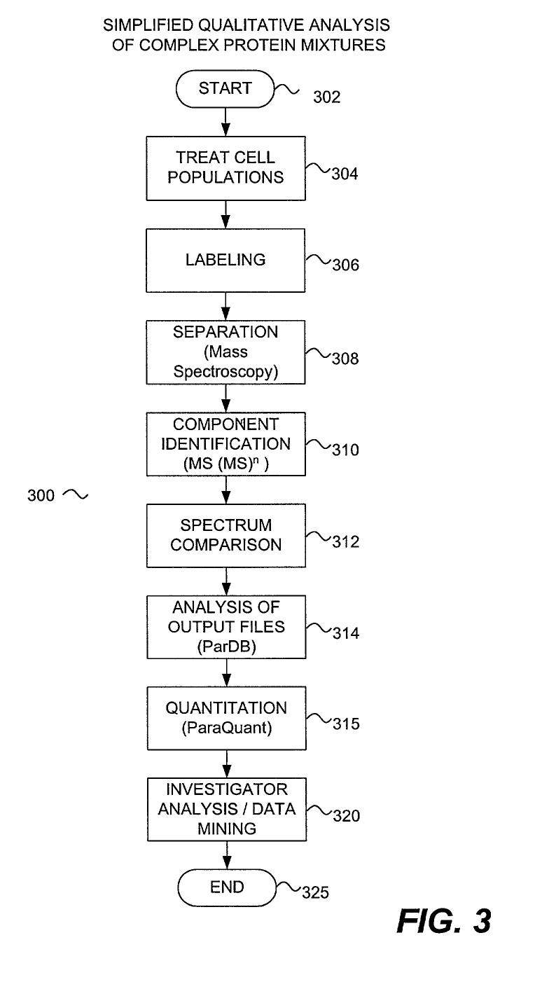

FIG. 3 further illustrates a method 300 for analyzing complex peptide mixtures using the aforementioned metabolic labeling or tagging methods to distinguish between different cell types or conditions. The process begins at a start state 302 and then moves to a state 304 wherein one cell population is treated differently from another cell population. Once the cell populations are treated, their peptides are isolated and labeled at a state 306.

As previously indicated, the labeling method may include metabolic labeling methods incorporating isotopes directly into the peptides or subsequent post-growth labeling methods with incorporate peptides of known sequence and mass into the peptides. Several examples of labeling peptides are provided below.

Following labeling, the peptides are then processed and separated by mass spectroscopy-based techniques at a state 308. In one embodiment, the mass spectroscopy-based techniques are preceded by the aforementioned MudPIT two-dimensional liquid chromatography methodology for separating the mixed-peptide population. Upon applying the mixed-peptide sample to the MudPIT column, the mixed-peptide sample is eluted off the column in a series of buffer washes (see Washburn et al., Nature Biotechnology, 19: 242-247 (2001) for additional information). Mass analysis of the eluted sample takes place as a plurality of independent “mass analysis snapshots” or scans which are performed sequentially over the time it takes for the mixed-peptide population to be eluted from the MudPIT column. In one aspect, mass analysis of the mixed-peptide eluate is performed at a rate of approximately 50 scans per second with approximately 9000 scans being acquired during the run of a typical mixed-peptide sample.

As the mixed-peptide population is eluted, the acquisition of sequential mass spectrum scans form a parent ion map or peptide elution profile for each of the peptides in the mixed population. Subsequently, peptide signatures or tandem mass spectrum are further generated by directing a portion of each eluted peptide through a second tandem mass analysis instrument to identify and characterize the peptides present in each parent mass spectrum scan. In one embodiment, the data analysis system 200 identifies the intensity of each of the peptide peaks within a particular mass spectrum scan or ion map and directs a tandem mass analysis to be performed for the most intense peaks using MS (MS)ⁿ. The resulting tandem mass spectrum or peptide signature is therefore generated for a limited number of intense peaks in the mass spectrum scan and the results of the scan are stored in the working database 226.

In a subsequent mass spectrum scan a similar process of identification of peak intensity is performed. The mass analysis system 200 determines if the most intense peaks have already been identified in the previous mass spectrum scan and, if so, selects new peaks with lesser intensities to perform tandem mass analysis on. Thus, the data analysis system 200 avoids performing redundant tandem mass analysis on peptides which are eluted over the time for which a plurality of mass analysis scans have been acquired to reduce the size of the data set which must be subsequently processed. Furthermore, by performing tandem mass analysis on a limited number of intense peaks, the data analysis system 200 improves the likelihood that each resolved peptide will undergo tandem mass analysis during the point in the elution where the peak intensity corresponding to the peptide concentration or abundance is of sufficient intensity to generate a useful high resolution tandem mass spectrum or peptide signature. Alternatively, tandem mass spectrum may be acquired for each peak within a particular mass spectrum scan or tandem mass spectrum may be acquired in another user-defined manner as desired. In this manner, data acquisition is facilitated, yet comprehensive information may be readily obtained to aid in the subsequent sequence identification.

When this method is applied to each mass spectrum scan acquired during the elution process, a plurality of tandem mass spectra are obtained which correspond to the plurality of resolved peptides 146. These spectra then undergo spectrum comparison at a state 312 by matching the spectrum from each peptide with the spectral database 250.

In the analysis of whole cell lysates it is not uncommon to identify in excess of 40000 individual spectral peaks corresponding to different resolved peptides which are to be desirably processed. The spectrum comparison state 312 likewise produces a very large number of peptide-correlated output files 260 to be subsequently processed by the data analysis system 200.

The data analysis system 200 facilitates the analysis of the peptide-correlated output files 260 by automating a number of the sorting and organizational tasks required to analyze the results returned from the spectrum comparison state 312 thereby reducing the burden to the investigator in identifying the components of the mixed-peptide population. In one aspect of this automation, the peptide data returned from the output files 260 is parsed and are stored to the working database 226. This process is explained more completely below.

Following analysis and storage of the spectral data, a subsequent quantitation is performed in state 315 to determine the relative abundance of the peptides originating from the different samples which have been mixed together at the onset of the analysis. During the quantitation state 315 the identity of each peptide that was subjected to a spectrum analysis is retrieved from the working database 226 and correlated with the mass spectrum peak heights and areas to determine the relative abundance of the identified peptide. Differential comparisons are additionally performed to correlate the expression of analogous peptides arising from the different peptide samples within the mixed population.

During the analysis of the peptide-correlated output files and quantitation steps, the data analysis system 200 may further employ advanced processes to identify spectral peaks which were not positively correlated by spectral comparison. For example, in the analysis of a whole cell lysate containing many thousands of individual peptide components, the mass spectra data 208 produced vary greatly from one to the next in terms of quality and information. In some instances, the spectral peak 146 may not possess sufficient signal strength to be positively identified by the component identification 145 and spectrum comparison process.

The data analysis system 200 provides functionality to correlate these weak or diminished spectral peaks 146 with analogous spectral peaks arising from the same peptide from a different peptide population within the sample. Thus, low abundance peptides can be positively identified based on an analogous peptide with a different label 122, 124. This feature of the data analysis system 200 improves the analysis of the peptide-correlated output files 260 and increases the sensitivity of the system in detecting and identifying low abundance peptides within the mixed-peptide population.

Upon completion of the analysis and quantitation of the mixed-peptide population, the resulting peptide identification and expression data is stored in the relational database 227 where it may be subsequently retrieved by the investigator and further utilized in a data mining operations state 320. The process 300 then ends at an end state 325.

The above mentioned peptide analysis method 300 desirably resolves the differentially labeled mixed-peptide population to produce a plurality of primary mass spectrum indicative of the individual components of the mixed population which are distributed based on their mass-to-charge ratio. Moreover, the mass analytical technique which produces the plurality of primary spectra possesses sufficient resolution capabilities to separate the mixed-peptide population into discrete and quantifiable units.

For each of the separated peptides, a subsequent tandem mass analysis is performed to generate a spectrum “signature” indicative of the peptide sequence of the separated peptide. The spectrum signatures are used as queries to interrogate the spectral database 250 which contains a plurality of previously associated peptide-correlated spectra. Typically, these queries produce a large number of results which must be correlated with the original spectrum signatures to verify the peptide sequence.

The peptide analysis method 300 comprises a series of instructions that determine the necessary associations between the spectrum signatures and the peptide-correlated spectra to identify each peptide in the mixed population. Furthermore, these instructions quantitate the individual peptides represented in the primary spectra and identify related peptides in the mixed-peptide population to assess differential expression in a manner that will be discussed in greater detail hereinbelow.

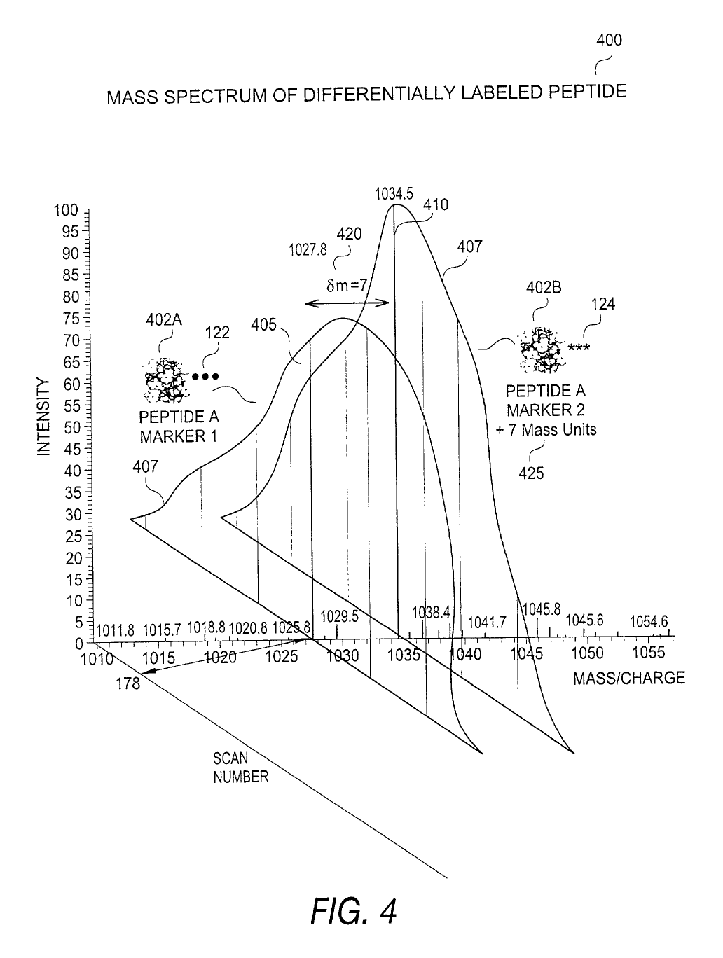

FIG. 4 illustrates a simplified mass spectrum scan diagram 400 for identical but differentially labeled peptides 402A, 402B. As previously described, the mass spectrum scan 400 comprises a plurality of individual mass analysis scans which are acquired over a designated time frame. Each individual mass analysis scan yields a snapshot of the peptides which are present in the portion of the eluate for which the mass analysis is conducted. By combining the results of the mass analysis scans an intensity curve 407 is generated for each peptide component of the mixed-peptide population. The intensity curve further represents the relative amount of the peptide component present at designated points in the mass analysis scan.

As shown in the illustrated embodiment, intensity measurements are assessed for a first peptide 402A containing a first marker and a second peptide 402B containing a second marker. At a designated scan number with a value of “178” (read from the z-axis of the mass spectrum scan diagram) the intensity for the first peptide 402A has an approximate value of “73” (read from the y-axis of the mass spectrum scan diagram) and an approximate mass-to-charge value of “1028” (read from the x-axis of the mass spectrum scan diagram). In a similar manner, at the same scan number “178”, the second peptide 402B has an approximate value of “98” and an approximate mass-to-charge value of “1035”. Using this method of data acquisition and comparison thus provides a means to compare the relative amounts of the two peptides 402A, B at any point where a mass analysis scan is performed. Furthermore, expression levels for each peptide 402A, B can be mapped over the time course of the elution and the maximal expression levels identified. In one embodiment, tracking of the maximal peptide expression levels as indicated by the intensity curves 407 is useful in improving the accuracy and sensitivity of peptides identification as will be discussed in greater detail hereinbelow.

A further feature of the data analysis system 200 resides in the mass differential created by analogous peptides whose sequence may be identical but whose mass-to-charge ratio differs as a result of the incorporated markers 122, 124. This mass differential represents a known or expected value which may be used to identify analogous peptides on the basis of the mass-to-charge distribution with or without supplemental peptide-correlated sequence information 260.

In an exemplary method demonstrating how the analogous peptide comparison feature may be applied, the data analysis system 200 identifies mass spectral scans comprising two or more peaks of interest where peptides 402A, B are compared. Assessing the mass-to-charge value a first peptide peak 405 associated with the first peptide 402A labeled with the first marker 122 yields a value of approximately 1027.6 mass-to-charge units while a second peptide peak 410 associated with the second peptide 402A labeled with the second marker 124 yields a peak at approximately 1034.5 mass-to-charge units. The mass-to-charge difference between the first peptide peak 405 and the second peptide peak 410 is observed as a displacement, or offset, of approximately “7” mass units 425. This displacement between the two peaks 405, 410 arises from the mass difference between the first and the second markers 122, 124 used to label each identical or analogous peptide 402A, B prior to mass analysis.

Thus, when analogous peptides derived from different biological samples or peptide populations 109A, B are labeled with discernable markers 122, 124 and these samples mixed, subsequent mass analysis scans resolve the peptides 402A, B into discrete peaks 405, 410 and form distinguishable intensity curves 407 that are separated by a distance proportional to the mass difference between the labels 122, 124. As will be shown in greater detail hereinbelow, this mass differential 420 may serve as a basis for separating and identifying analogous peaks in the mixed-population peptide sample. Additionally, the mass differential 420 may be used to identify peptides whose relative concentration within the mixed-peptide population is too low to be positively correlated with known peptide sequences within the spectral database 250. Further details describing aspects of the differential labeling method used to discriminate analogous peptides based on the mass differential are described in the section entitled “Peptide Labeling Methods”.

Differential labeling of the mixed-population of peptides in the aforementioned manner provides a means for identifying peptides derived from each peptide population that are mixed prior to mass analysis. The separation distance of the exemplary analogous peptides illustrated in the mass analysis scan 400 is proportional to the mass of the markers 122, 124. This mass differential 420 created between the labeled analogous peptide is used by the data analysis system 200 to validate that two peptide peaks found in the primary spectrum are analogous. Without a differential mass label, analogous peptides from each sample would have identical mass-to-charge ratios and thus be indistinguishable from one another. The resulting spectrum would therefore lack any discernable differences which could be used to identify analogous peptides and difficulties would arise in determining how much peptide was being contributed from each cell or tissue type under comparison.

Additionally, the mass differential created by the markers 122, 124 may be used by the data analysis system 200 to determine the region of the primary spectrum which should be scanned for analogous peptides rather than comparing each spectrum signature with all others produced by peptides of the primary spectrum scans. As will be subsequently shown, this feature is useful in dividing the comparison and quantitation calculations into smaller subsets that may be operated on in parallel to improve acquisition of experimental results.

1. Correlation of Mass Spectral Information

Matched Peptide Correlation

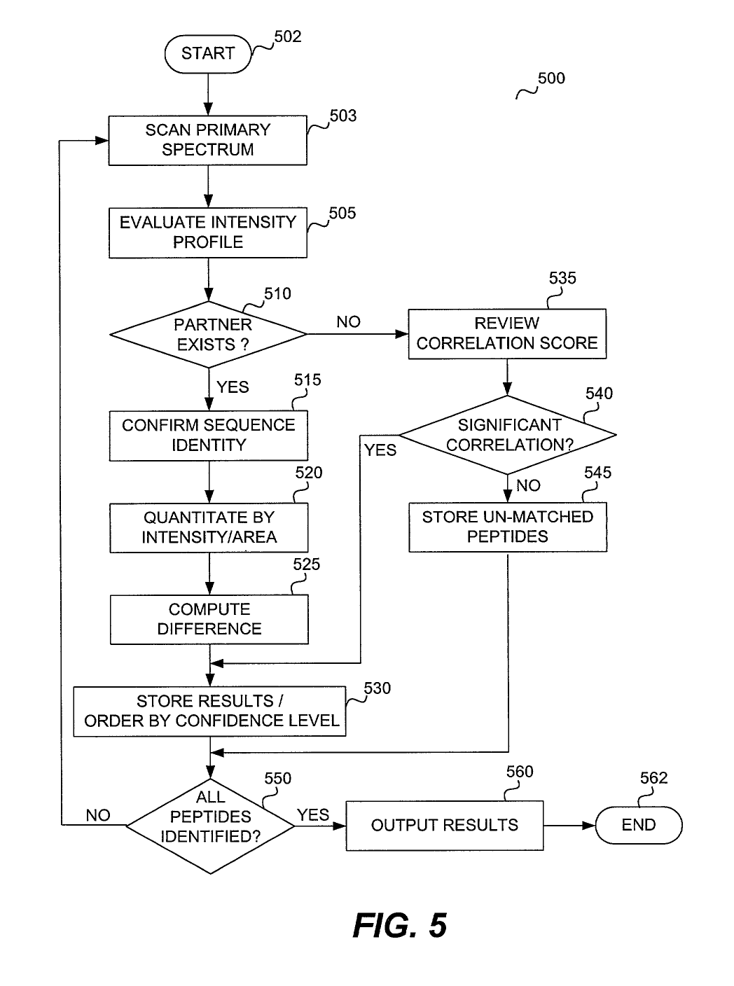

FIG. 5 illustrates one embodiment of a correlation process 500 used by the data analysis system 200 to identify and correlate peptide peaks corresponding to resolved peptides 146 obtained by mass analysis. The process begins at a start state 502 and proceeds to a state 503 where scanning of the primary mass spectra 208 takes place. The primary mass spectra 208 comprises a plurality of mass analysis scans corresponding to sequential time points in the elution of the mixed-peptide population. Each mass analysis scan further corresponds to an ion map, snapshot, or image of the proteins which are present in the eluate during the time at which the mass analysis scan was performed.

As will be described in greater detail in subsequent figures, eluted peptides that are detected in the primary mass spectra 208 are further analyzed be tandem mass analysis to generate peptide signatures characteristic of each of the peptide sequences. The collection of signatures are then used to query the spectral database 250 to aid in the identification of the peptides by correlation with tandem mass analysis spectrum of known sequences.

In one embodiment, peptide matching against the spectral database 250 takes place in a batch process where peptides associated with the first discernable population are processed and the results stored in the working database 226. Subsequently, peptides associated with the second discernable population are then processed and results similarly stored in the database 226. The data analysis system 200 may recognize peptides arising from each peptide population by identifying the characteristic mass difference between the peaks in the mass spectrum scans.

The results 260 obtained from the queries of the spectral database 250 include information which aids in the identification of each peptide sequence. One component of the query result 260 comprises a correlation result which identifies a known peptide sequence that is likely to be similar to the experimental peptide sequence from which the query was formed. Additionally, a correlation score may be used to indicate the degree of certainty of the correlation result. A high correlation score is indicative of a high degree of certainty for the identification of the experimental peptide sequence. In a similar manner a lower correlation score is indicative of a lesser degree of certainty for the identification of the experimental peptide sequence. The value of the correlation score is desirably used in conjunction with the mass-differential created by the peptide markers 122, 124 to identify the peptide components of the mixed-population and determine the proteonomic differences as will be described in greater detail hereinbelow.

The process of peptide correlation 500 continues in a state 505 where the elution profile for each of the peptides is assessed. During this state 505, the peptide peak intensity across the plurality of mass analysis scans obtained during the time course of the elution is evaluated to produce an intensity curve indicative of the relative abundance of the protein during the elution. Using the information obtained from the intensity curve, quantitation of the peptide can be made by evaluating the summation of the peak intensities for all mass analysis scans along the intensity curve where the peptide is found. Additionally, in evaluating the intensity profile 505 for each peptide, the data analysis system 200 further identifies the a time frame of the elution corresponding to a particular mass analysis scan where the intensity of the peptide is maximal and stores this value in the working database 226 for use in identifying analogous peptides labeled with different markers 122, 124.

In a decision state 510, the correlation process 500 scans each mass spectrum scan incrementally and upon identifying a peptide, determines if a corresponding analogous peptide or partner exists in the spectral vicinity. In one aspect, corresponding analogous peptides can be identified by scanning for peaks displaced by an appropriate mass distance, dependent on the marker or label 122, 124 used to tag the mixed-peptide population. For example, as shown in the previous illustration, the correlation process 500 identifies the first peak 405 and scans the primary mass spectrum in the regions that are displaced approximately 7 mass units away from the first peak of interest to determine if the second peptide peak 410 is present.

While in the decision state 510, if the data analysis system 200 determines that the identified peptide possesses a potentially analogous partner, as indicated by the presence of the second peak 410 with the appropriate mass difference, the process 500 proceeds to a state 515 where the sequence identity of both peaks 405, 410 is confirmed. Alternatively, if the data analysis system 200 determines that the identified peptide does not possess and analogous partner, the process 500 proceeds to a state 535 where the correlation score for the identified peptide is reviewed (see section below entitled Un-matched Peptide Correlation).

In the case of identified peptide partners where the process 500 has reached the sequence confirmation state 515, the peptide sequences for each identified peptide are confirmed using information obtained from the MS (MS)ⁿ analysis and subsequent peptide-correlated output files 260. During the sequence confirmation state 515, the data analysis system processes correlate analogous peptides by both sequence-related information, as well as, expected mass differences to establish the relationship between the two discernibly labeled peptides with a high degree of certainty.

The sequence confirmation state 515 additionally incorporates an intensity scanning feature that is useful in identifying peptides of low abundance or whose tandem mass analysis scans produce inconclusive results. Using this feature, the data analysis system 200 may proceed identify a different region of the intensity curve 407 for the particular peptide of interest which is associated with a different mass analysis scan. Typically, the region of the intensity curve 407 selected corresponds to a region where the peptide is present in greater abundance (as indicated by a higher intensity). The data analysis system 200 may then review the results of the tandem mass analysis taken in this higher intensity region and any spectral database queries performed for the peptide to improve the positive identification of peptide sequences and facilitate analogous peptide identification. Additionally, when using this method, the data analysis system 200 is able to acquire useful peptide sequence information from other regions or mass analysis scans which may be correlated with the region where the tandem mass analysis of the peptide produced inconclusive results. Thus, if one peptide is below the threshold of resolvability of the MS (MS)ⁿ analysis at a particular time point or if the peptide-correlated output files 260 do not imply a clear sequence identity, the data acquisition system 200 may utilize the plurality of mass analysis scans and tandem mass analysis taken over different times to better resolve the each peptide sequence and confirm the sequence identities between two analogous peptides.

Following the confirmation state 515, the process 500 proceeds to a state 520 where peak or intensity curve areas for analogous peptides are determined. As previously indicated, these calculations are representative of the amount of peptide present in the mixed-population sample and may be used to determine changes in peptide expression by computing the difference between analogous peptides. As will be described in greater detail in subsequent illustrations and discussion, the analysis of the peak area and intensity curves desirably employs a specialized method for identifying and resolving each peptide associated data set to improve the quantitation and integration of the area defined by the bounds of the data set. The quantitation methods used in this state 520 desirably provide improved accuracy in assessing the relative abundance of each peptide in the mixed population and aid in identifying proteomic differences in the cells or tissues under comparison. Additionally, the quantitation methods may be used to identify peptide abundance at specific times during the elution of the peptide (corresponding to individual mass analysis scans), as well as, across the overall time frame for which the elution of the peptide takes place (corresponding to the plurality of mass analysis scans).

After quantitating the analogous peptides the process 500 proceeds to a state 525 where the peptide abundances or concentrations are compared. In this state 525, differences in abundance between the analogous peptides are identified by calculating the difference between the quantities of peptides determined in state 520. This information provides valuable insight into proteomic differences between analogous peptides in the mixed-population and serves as an indicator of differences in expression or regulation of the peptides as will be shown in greater detail in subsequent figures.

The process 500 then proceeds to a state 530 where the results of the aforementioned calculations are stored within the relational database 227. As will be appreciated by one of skill in the art, the relational database 227 may comprise a plurality of tables or fields which may be interrelated via associations. These associations are used to generate meaningful queries, such as those used to produce reports, which display the associations between analogous peptides in the cell or tissue samples. The use of the relational database 227 also provides a means of interrelating data obtained from a plurality of different mass analysis experiments and aids in data mining operations used to evaluate and associate differential peptide expression in various conditions and biological samples of interest. In one aspect, the peptide calculations may include a confidence score which is used to order the results based on the degree of confidence with which the peptide identification and/or comparison is made. Furthermore, other identifiers or relationships can be stored in the relational database 227, including information that correlates the identified peptides to other resolved peptides within the mass analysis spectrum. As previously discussed, at least a portion of this information may be obtained from other bioinformatic databases 254 which are queried by the data analysis system 200 and the results stored with the associated peptide sequence and quantitation results.

Un-matched Peptide Correlation

In those instances where the correlation process 500 reaches the decision state 510 and determines that the resolved peptide does not possess an identifiable partner (analogous peptide), the process 500 proceeds to a state 535 wherein the correlation score of the peptide comparison is reviewed. In this state 535, results (in the form of peptide-correlated output files) are obtained from queries of the spectral database 250 (corresponding to the tandem mass analysis spectrum of the resolved peptide). The process 500 proceeds to a decision state 540 wherein an assessment of the results of the spectral database queries is made. In this state 540, the data analysis system 200 identifies if significant correlation exists between the resolved peptide and any mass analysis spectrum in the spectral database 250. If a significant correlation is determined to exist between the resolved peptide and an entry in the spectral database 250, the process 500 moves to the state 530 wherein the putative sequence of the resolved peptide is stored along with an indicator of the relative confidence level of the correlation.

If a significant correlation is not found at the decision state 540, the process 500 moves to a state 545 wherein novel or un-matched peptides (which are identified by a lack of significant correlation with existing entries in the spectral database 250) are stored in the relational database 227 with an appropriate identifier denoting that the peptide is unidentifiable or possesses a low correlation score indicating that the resolved peptide's sequence was not known with certainty.

Upon storing the results for analogous or identifiable peptides in state 520 or storing the results for peptides with little or no sequence homology in state 545 the process proceeds to a decision state 550 and determines if all resolved peptides have been assessed. If additional peptides remain to be correlated, the process returns to the scan spectrum state 503 and performs the indicated functions. When all peptides have been processed in the aforementioned manner, the process 500 proceeds to a state 560 where the results of the analysis may be output to the investigator. In this state 560 data summaries and automated calculations may be made which are subsequently output in a user-defined manner to provide the investigator with one or more flexible reports of the experimental results including peptide sequence identifications and correlation, differential expression analysis of analogous peptides, novel peptide identification, and confidence level assessments for the peptide correlations. Finally, the process proceeds to an end state 562 completing the peak analysis process 500.

The aforementioned correlation process 500 therefore implements a method to identify each peptide in the primary mass analysis spectrum and, if possible, associate analogous peptides labeled with the different markers 122, 124. Furthermore, the correlation process 500 quantitates the relative abundance of each peptide and may use this information to aid in the determination of proteomic differences. Proteomic differences between analogous peptides are subsequently used to identify changes in peptide expression or abundance corresponding to the treatment or condition which the cells or tissues were exposed to and provides an important tool for investigators to use in assessing complex peptide populations and biological processes.

As will be subsequently described in greater detail, the amount of data which must be analyzed during the correlation process is quite large. As a result, the time required to perform the analysis can take many hours to complete. Although it is possible to perform the necessary calculations on a single computing device, the correlation process 500 is desirably implemented in a clustered environment to improve computing performance and yield results more quickly. In the clustered computing environment the correlation process 500 is performed in a parallel computational manner where the work of identifying and comparing peptides is subdivided and distributed across a plurality of computing devices configured to process the spectra in a distributed manner.

2. Exemplary Mass Spectra Data

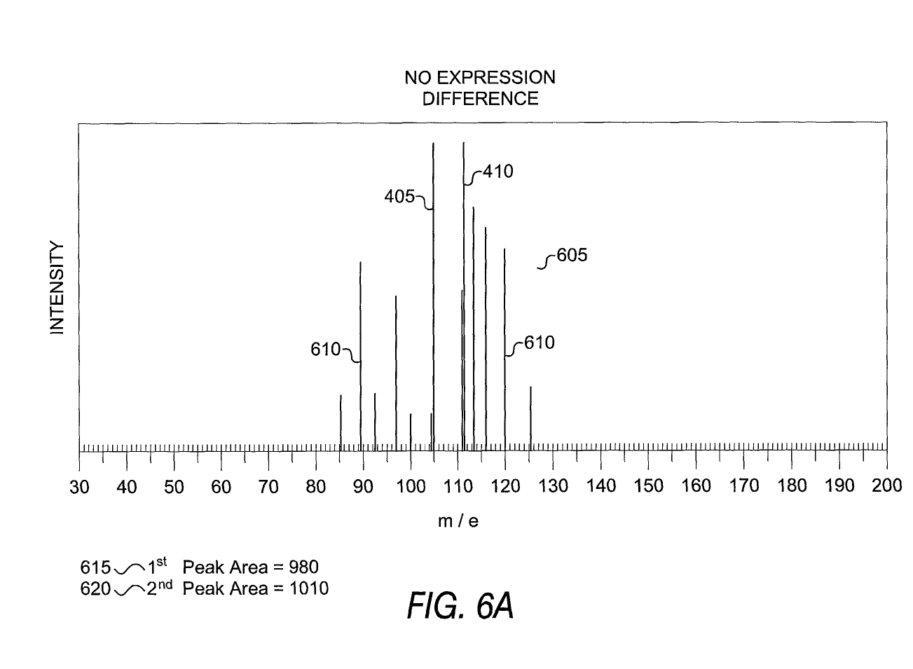

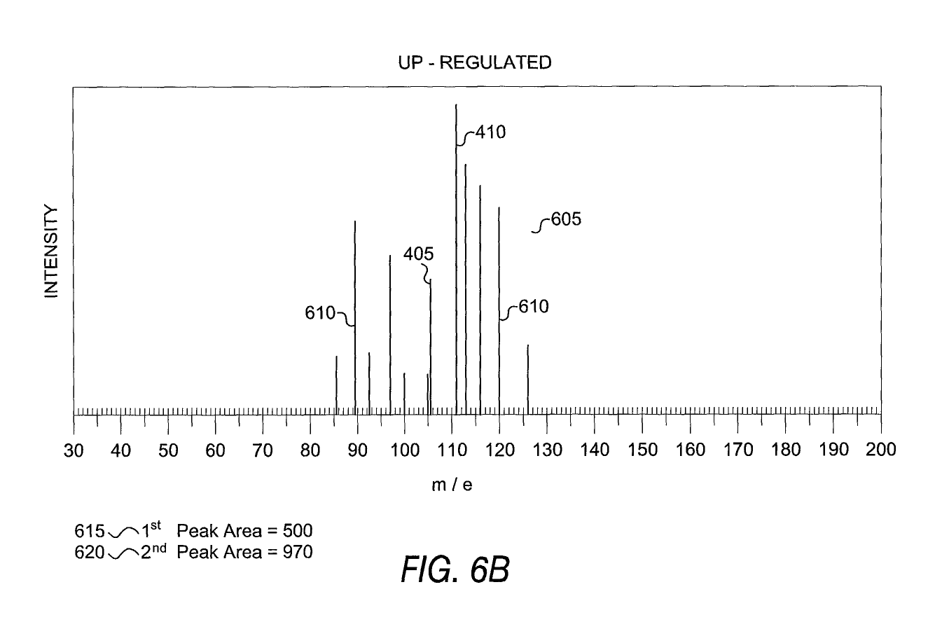

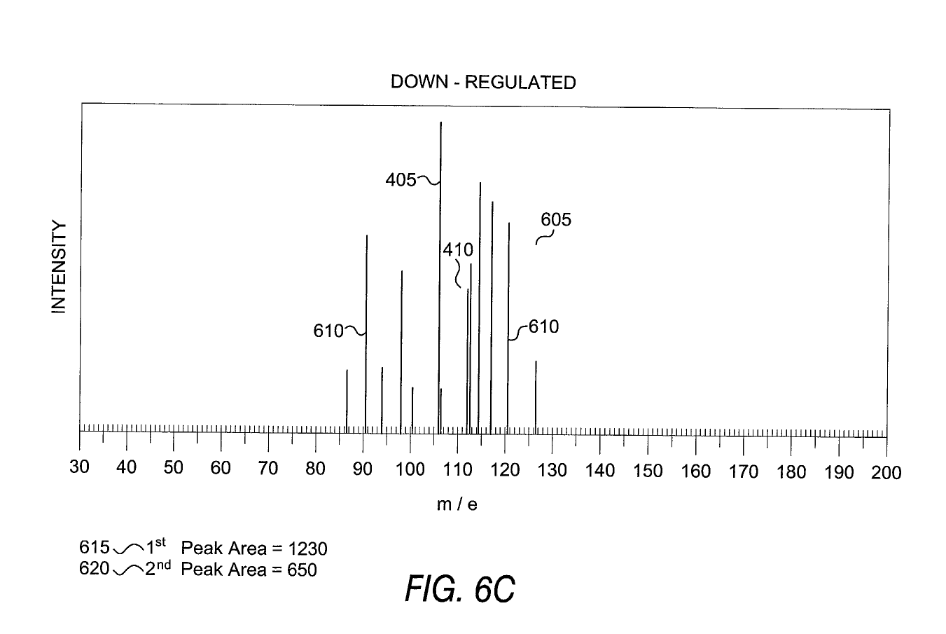

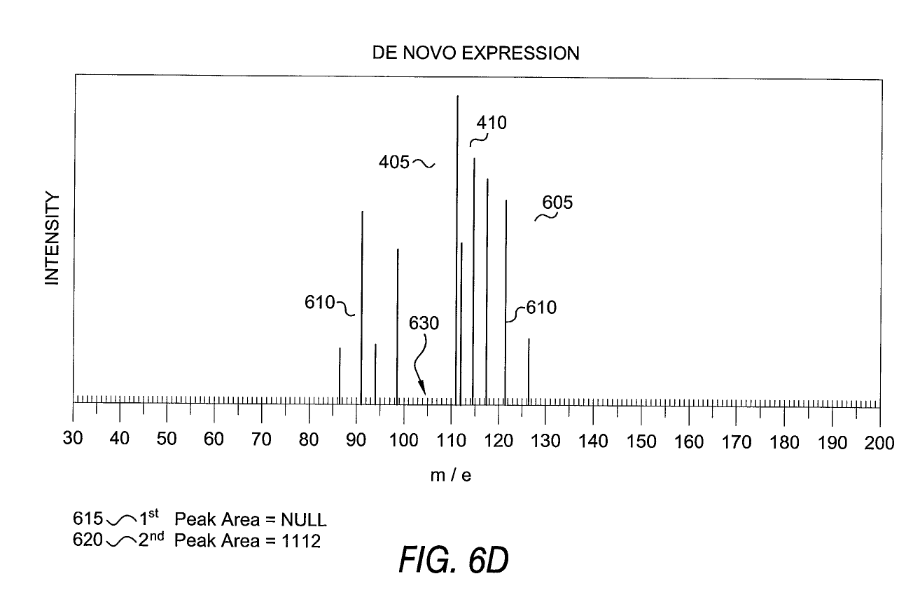

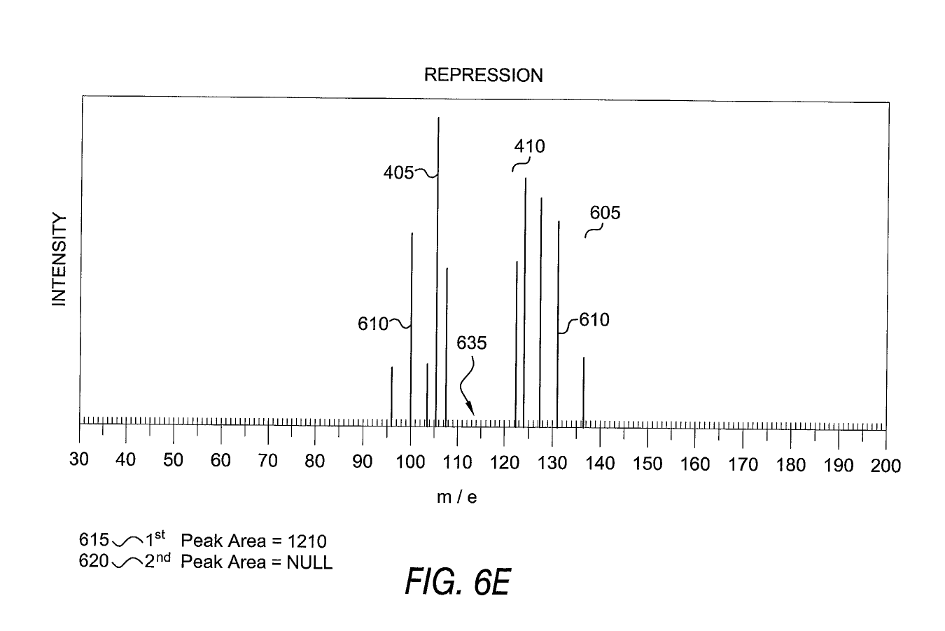

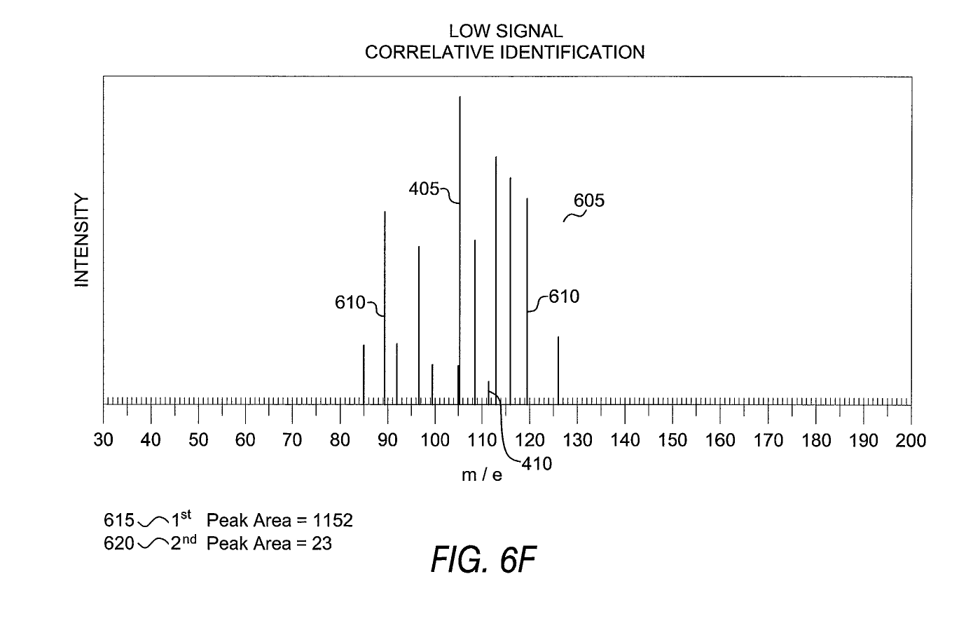

FIGS. 6A-6F illustrate a collection of exemplary mass spectrum scans depicting states of differential expression which may be identified by the data analysis system 200. In each figure, a collection of peaks 605 is shown with each peak indicative of a peptide component of the mixed-population that has been separated by mass analysis. The correlation process 500 subsequently identifies a first peak 405 and a corresponding partner or analogous second peak 410. Confirmation of both the appropriate mass difference (seven mass units in the illustrated embodiment) and the tandem mass spectrum (not shown in the illustration) results in the comparison process 500 identifying these peaks 405, 410 as analogous and having the same peptide composition with different labels or tags. Confirmation further prevents other peaks 610 in the mass spectrum from being inappropriately associated with the two analogous peaks 405, 410. As previously indicated, upon confirming the relationship between the peaks 405, 410 the data analysis system 200 performs a quantitation of peak areas and intensity values to determine the relative amount of peptide within the sample and compares these values to one another to determine proteomic differences.

In FIG. 6A, a first peak area 615 is associated with the first peak 405 and has a value of “1000” with a second peak area 620 associated with the second peak 410 also having a value of “1000’. A calculation of the difference between the peak areas 615, 620 of the analogous peaks 405, 410, results in a difference value of “30” (1010-980=30). This difference in peak areas is representative of resolved peptides that do not possess substantially altered differences in expression.

FIG. 6B illustrates an exemplary mass spectrum scan for a labeled peptide having an up-regulated expression pattern. Similar to the manner of identification and confirmation as described above, the data analysis system 200 identifies the first peak 405 and the second peak 410 as analogous based on their mass difference and tandem mass spectrum. In the case of up-regulated expression the first peak 405 possesses a substantially reduced peak area 615 compared to the area 620 of the second peak 410. The data analysis system therefore recognizes this pattern of expression as being up-regulated when comparing the quantity of peptide 402 labeled with the first label 122 relative to the quantity of peptide 402 labeled with the second label (see FIG. 4). Conversely, peptide down-regulation as illustrated in FIG. 6C, may be determined by the data analysis system 200 when the first peak 405 possesses a substantially increased peak area 615 relative the area 620 of the second peak 410.

FIG. 6D illustrates an exemplary mass spectrum scan for a labeled peptide exhibiting de-novo expression. As shown in the illustrated embodiment, the lack of the first peak at the expected position 630 in the mass spectrum in addition to the presence of the unpaired second peak 410 is indicative of only the peptide population labeled with the second label 124 containing the indicated peptide. In one aspect, an expression pattern where an unmatched peak is present in the mass spectrum scan may indicate de-novo expression of a peptide which is potentially of significant interest to investigators.

Alternatively, FIG. 6E illustrates and exemplary mass spectrum scan for a labeled peptide exhibiting repression. As shown in the illustrated embodiment, the presence of the first peak 405 in addition to the lack of a corresponding or paired second peak at the indicated position 635 may identify a peptide that is found only in the first peptide population labeled with the first label 122.

In the case of unpaired peptides encountered in the mass analysis, further characterization by the correlation process 500 may be performed to determine if there is significant correlation between the tandem mass spectrum of the peptide with those in the spectral database 250. This information is useful in identifying peptides with novel sequences, as well as, flagging those peptides whose level of expression changes dramatically when comparing the two peptide populations.

FIG. 6F illustrates an exemplary mass spectrum where low signal strength in the second peptide peak 410 may be correlated with a positive identification of the first peptide peak 405 to yield a putative identification of an otherwise unidentifiable peptide. As shown in the illustrated embodiment the second peak possesses a peak area 620 indicative of a peptide whose low abundance prevents identification by tandem mass spectroscopy. The peak analysis process 500 however is able to associate the second peak 420 with the first peak 405 on the basis of the mass differential. In the absence of confirming tandem mass spectroscopy data, this type of identification can be important in identifying peptides which fall below the threshold of detectability of the instrumentation in one mixed peptide population but are readily detectable in a second peptide population.

The aforementioned exemplary mass spectra demonstrate an overview of how peptide expression between two or more samples may be correlated to identify differences in peptide expression. Based upon the identification of analogous peaks 405, 410 that are appropriately displaced by incorporation of the markers 122, 124, the data analysis system quantitates relative amounts of peptide expression and readily compares these values in the cells or tissues under study. Comparison of peptide expression in this manner provides important insight into changes or alterations in differential peptide expression and may identify peptide expression states of interest.

Another useful feature of this system relates to the aspects of analysis whereby the majority of peptides contained within a cell or tissue of interest may be analyzed simultaneously. This feature provides a global assessment of peptide expression which is in many cases necessary to better understand important biological relationships between related peptides and pathways.

A further feature of this system relates to the simultaneous analysis of two or more peptide populations within the sample mixed population sample. Analysis within the same sample desirably reduces problems associated with background, noise, and spurious or stray data which might otherwise confound differential expression analysis. These problems are commonly found in experimental mass analysis where each peptide population is evaluated independently of one another and increases the difficulty in positively and accurately identifying and associating peptides across multiple sample sets.

In one embodiment the aforementioned mass spectra depict mass spectrum scans taken at particular time intervals during the elution of the mixed peptide population. As will be appreciated by those of skill in the art, the principles and methods for mass spectral analysis to identify proteomic differences can additionally be carried out using the intensity curves 407 formed from the aggregate of the plurality of mass spectral scans taken over a designated time interval. In this embodiment, peptides are quantitated and compared based on the total peptide concentrations within the mixed population sample. This method of proteomic analysis desirably normalizes the difference analysis over the plurality of mass analysis scans and reduces quantitation errors which might arise from slight differences in elution at particular times during the mass spectrum acquisition process. In a manner similar to that used in comparing analogous peptides in the mass analysis scans, the intensity curves 407 may be used for analogous peptide comparison. Thus, proteomic differences, peptide identification, and peptide quantitation can be performed both on individual mass analysis scans and on the intensity curves as a whole.

3. Quantitating Sample Differences in Parallel

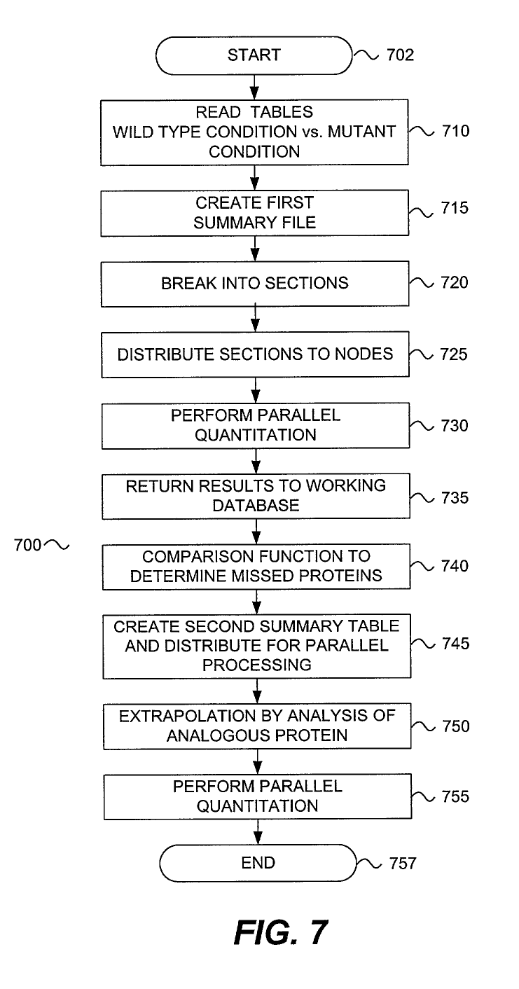

FIG. 7 illustrates a flow diagram used by the data analysis system 200 to identify and quantitate the chromatographic scans of the mass spectra associated with the differentially labeled peptides. The process of identification and quantitation is a computationally demanding task as there are typically thousands of individual scans which must be analyzed to associate and identify analogous peptides. Furthermore, the relative abundance of the peptides represented in each scan must be evaluated and correlated between analogous, but differentially labeled, peptides. In the illustrated embodiment parallelization of tasks is used to improve computational performance by distributing the computational work to be performed among a network of computers. Although, the data analysis system 200 can be readily adapted to process the mass spectra in a non-parallel manner, such a system may lack the improvement in performance gained by distributing the computational workload over a number of computers within a cluster.

Parallel computational methods utilize a plurality of independent microprocessors and/or computers to solve complex problems in a more rapid manner than can be accomplished using a single computer or processing device. In a parallel architecture, computers are typically interconnected by networking connections forming a plurality of nodes within a clustered environment which exchange information and operate in a coordinated manner using a parallel computational language. The parallel computational language is designed to implement specialized programming and communication requirements necessary for solving problems in a distributed manner. Examples of commonly utilized parallel computational paradigms include Parallel Virtual Machine (PVM), Message Passing Interface (MPI), load sharing facility (LSF), or other similar methods to create programming instructions and processes that can be simultaneously executed on a plurality of computational devices to solve problems rapidly and efficiently. For additional details relating to these parallel implementations the reader is directed to the following references: Pvm: Parallel Virtual Machine: A Users' Guide and Tutorial for Networked Parallel Computing, Al Geist, MIT Press (1994); Using Mpi: Portable Parallel Programming With the Message-Passing Interface, William Gropp, Ewing Lusk, Anthony Skjellum, MIT Press (1999); Parallel Programming: Techniques and Applications Using Networked Workstations and Parallel Computers, Barry Wilkinson, C. Michael Allen, Prentice Hall (1998).

The data analysis system 200 typically stores the necessary information about each chromatographic peak and intensity curve 407 in one or more tables of the working database 226. This information includes the results 260 of the sequence queries directed towards the spectral database 250. As previously discussed, these queries are created by the data analysis system 200 using the tandem mass spectra 147 generated from each resolved peptide 146. The resulting peptide-correlated output files 260 obtained by comparison of the tandem mass spectrum 147 against the spectral database 250 provides a preliminary basis of knowledge and information used to evaluate the sequence and composition of the resolved peptides 146. As the data analysis system 200 receives the peptide-correlated output files 260 the associated information is stored in the aforementioned database 226 where it is subsequently processed in a manner that will be described in greater detail hereinbelow.

Additional information which may be stored in the database 226 includes information identifying chromatographic peak or intensity curve areas, mass-to-charge ratios, peptide-correlated data output, or other information useful in associating or pairing the differentially labeled peptides from the mixed-population. In one aspect, this information is stored in tables or arrays within the database 226 to facilitate cataloging, sorting, querying, and storage/retrieval of the information used to determine the peptide sequences and proteomic differences in the biological samples. These tables may additionally be arranged according to the results of the tandem mass spectroscopy obtained for each condition, cell treatment, peptide-population, and/or label and are used to distinguish between the peptides in the mixed-population that underwent mass analysis.

In an exemplary differential analysis comparing a wild-type peptide population with a mutant or treated peptide population, two tables are generated and compared which correspond to a first table containing information relating to the wild-type condition and a second table containing information relating to the mutant condition.

Thus, the process 700 for identification and quantitation of the chromatographic peaks and intensity curves proceeds from a start state 702 to a state 710 where the data analysis system 200 reads data from the tables and acquires information contained in the fields of interest. The process 700 then moves to a state 715 wherein a first summary file is created containing information necessary to perform the peptide identification and quantitation analysis, while removing unnecessary information which might otherwise reduce the performance of the parallel processing routines. The process then proceeds to a state 720 where the quantitation summary is broken into a plurality of data sub-sections 720 to divide the data into smaller pieces which may be operated upon individually. The creation of data subsections at the state 720 additionally facilitates the distribution of the experimental data across the plurality of nodes improving the ability to perform the identification and quantitation in parallel.

The identification of the peptides commences when the data sub-sections are processed in a state 725 and distributed across the plurality of nodes within a computing cluster. After receiving the data sub-sections, the process 700 proceeds to a state 730 where each node quantifies the chromatographic peaks and intensity curves. The quantitated data is then sent back to the database 226 in state 735 where results are captured and collated.

After the initial quantification is complete, the process 700 moves to a state 740 wherein a comparison function is performed to identify any chromatographic peaks whose tandem mass analysis spectrum can not be correlated with an associated entry in the spectral database 250, thus indicating that the peptide may not be identified accurately.

Subsequently, the process 700 proceeds to a new state 745 where the chromatographic peaks and their associated information fields are used to build a second summary table which is redistributed for parallel processing in the aforementioned manner. The process 700 then moves to a state 750 wherein the peaks and intensity curves 407 are requantified by extrapolation to improve the level of confidence of the identification of the peptide.

The extrapolation state 750 is performed by identifying the paired or analogous peptide which reside an appropriate number of mass units away from the unidentified peptide (mass shift), depending on the differential mass labeling technique chosen. During state 750, differentially labeled peptides which are analogous (having similar sequences but different labels and derived from different biological samples) are identified based up knowledge of the expected mass differential between the markers 122, 124 used to label the two or more peptide population being compared. Following identification, the process advances to an end state 757 where quantitation is completed and the results stored in the relational database 227.

During the identification and correlation of analogous peptides, the data analysis system may proceed through a first collection of resolved peptides whose sequence identity are confirmed by spectral database 250 comparison. Furthermore, these peptides may be associated with partner (analogous) peptides whose mass-to-charge ratio is displaced or offset from that of the resolved peptide. The data analysis system 200 confirms the relationship between the resolved peptide and the analogous peptide by verifying that the mass difference between the two peptides occurs with an expected value dependent upon the markers 122, 124 incorporated into the peptide populations. Furthermore, the data analysis system 200 may confirm the peptide-correlated output files 260 for the two peptides are consistent with the peptides having the same sequence. In this manner, the data analysis system 200 is able to identify and associate peptides with similar sequences that have been derived from different cells, tissues, treatments, and/or conditions. The results of this identification procedure are then stored in the aforementioned database 226 where they may be formatted, queried, and presented in user-defined manners.

For those peptides whose sequence cannot be identified with certainty based upon the peptide-correlated output file 260, a subsequent identification process may be attempted in order to maximize the chances for identifying the peptide sequence. In this process the data analysis system 200 reviews the primary mass analysis scans and identifies the unknown peak or intensity curve. Subsequently, the data analysis system 200 scans the mass-to-charge region of the spectra coinciding with a region where an analogous peptide (containing the different marker) might be expected. If an analogous peptide peak or intensity curve is identified, the data analysis system 200 may correlate the tandem mass spectrum of the peptides and determine if the spectra are similar enough to associate the sequence information of the analogous peptide with that of the unidentified peptide.

In certain instances, the tandem mass spectrum produced for the peptide is of low resolution or quality. This is typically due to a low abundance or concentration of the peptide in the eluate which was used to generate the tandem mass spectrum. The resulting low resolution tandem mass spectrum may contribute to a low confidence sequence match with the spectral database 250. To improve in the identification of peptides which posses such low resolution spectra, the data analysis system 200 may scan through the intensity curve of the peptide and locate an area or region where the peptide intensity is maximal. The data analysis system 200 may then assess the tandem mass spectrum for the peptide taken in this region to improve the quality or resolution of the spectrum which may be subsequently compared against the spectrum database 250. This process desirably improves sequence identification and increases the confidence of matches. Upon identifying the sequence of the peptide in the region of maximal intensity, the data analysis system 200 may correlate this information with the mass spectrum scan having low peptide abundance or concentration to identify each peptide with greater accuracy and sensitivity.

Furthermore, the intensity curve scanning technique described above can be applied to instances where analogous peptides are difficult to determine in a particular mass spectrum scan. Using this method, the data analysis system 200 may scan peptide intensity curves for both the peptide of interest and the putative analogous peptide to identify areas of maximal intensity. In these regions of maximal intensity, the tandem mass spectra can be assessed to improve the accuracy and sensitivity of the identification of each peptide. The results of the identification can then be correlated with one another to aid in identification of the analogous peptides and proteomic differences.

Peptides which are identified using the intensity curve scanning methods are requantified and the results summarized and returned as before. Those peptides which cannot be conclusively identified are flagged during the quantification procedure and the results returned to the working database 226 where they may be summarized independently. Unidentified peptides are significant in that they may represent novel peptides whose expression cannot be correlated with information in existing spectral databases and are typically of interest to investigators.

The aforementioned method 700 for identifying and quantitating data uses parallelizable tasks to improve the ability of the data analysis system 200 to process the large numbers of peptides that might be found within an entire organism or tissue sample. To improve the efficiency of processing, each parallelizable task is desirably divided in such a way so as to associate the specific data files and information required for analysis of the resolved peptides 146. This association of information improves the computational efficiency of identifying and quantitating the resolved peptides and reduces the amount of data that must be transferred between nodes.

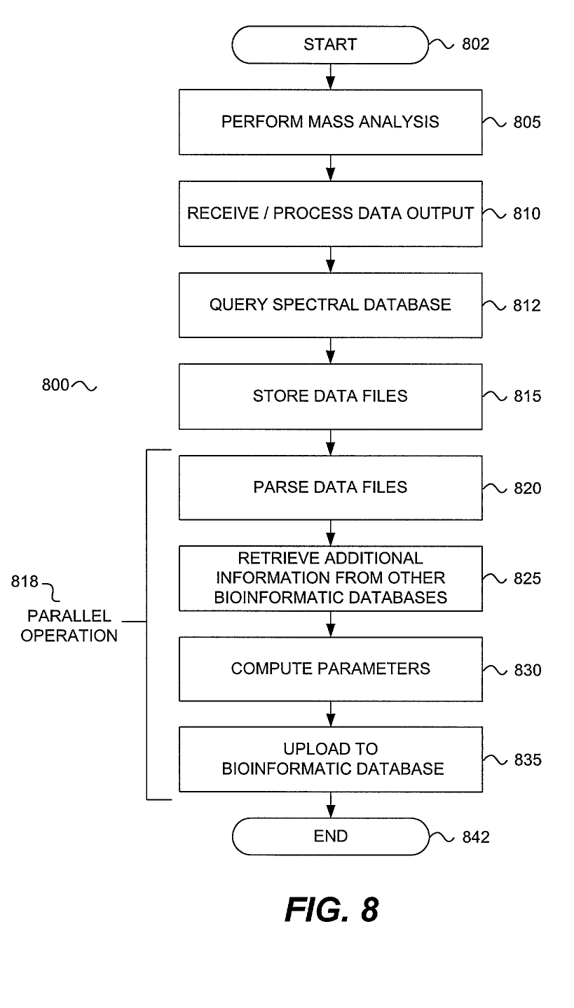

FIG. 8 illustrates a flow diagram of a process 800 in which the data output comprising the mass spectrum information 208 is analyzed by the data analysis system 200. Beginning in a start state 802 the process proceeds to a state 805 where analysis of the labeled mixed-peptide population 130 takes place. In this state 805, the primary mass analysis is performed to separate the components of the mixed-peptide population 130. Furthermore, the subsequent tandem mass analysis is performed on each resolved peptide to generate the unique mass spectrum which is dependent on the sequence or composition of the peptide.

The resulting spectral information including the primary mass spectrum and the plurality of tandem mass spectra, as well as, associated data and information produced by the instrumentation 205 are received by the data acquisition module 220 of the data analysis system 200 in a state 810. In this state 810, the spectral data and information may be re-arranged, cataloged, formatted, or otherwise processed into a form suitable for storage in the working database 226. Additionally, the data processing module 225 of the data analysis system 200 may associate the spectral data and information with informational identifiers such as investigator-input descriptions of the experimental conditions, cell types, sample quantities, markers used, and other information which is useful in identifying and assessing the spectral data. Processed spectral data and information is stored in the database 226 according to an organizational schema that separates the data into component parts and stores it within the database 227 in a plurality of data tables and fields as will be subsequently illustrated in greater detail.

Upon completion of the aforementioned database population, the process 800 proceeds to a state 812 where the spectral database query is prepared. In this state 812 the data processing module 225 retrieves information from the database 226 including experimental tandem spectra and associated information from one or more of the resolved peptides. This information is further formatted and organized to form a query command or file which is submitted by the communications module 235 to the spectral database 250. In one embodiment, the data analysis system 200 forms and submits a combined or composite query in which a plurality of spectrum and information to be analyzed is submitted as a batch file to be processed by the spectral database 250. Additionally, the spectrum and information can be reviewed by the investigator and customized queries developed which are submitted in a manner similar to the automated queries generated by the data analysis system 200.

Queries which are received by the spectral database 250 are then compared against the plurality of mass spectra with known peptide sequences. As previously discussed, the results of the query comprise one or more peptide-correlated output files 260 which contain information indicating the correlation between the experimentally resolved peptide and those contained in the spectral database 250. The output files 260 are sent back to the data analysis system 200 in a subsequent step 815 where they are processed and stored in the database 226.

In an experiment where many thousands of peptides are simultaneously assessed, the amount of information contained in the uploaded output files 260 is quite large. Furthermore, each output file 260 typically comprises numerous fields and types of information which are associated with the analysis and identification of each peptide. In order to more efficiently complete the analysis of the mixed-peptide population, the data analysis system 200 desirably performs a number of steps of the analysis in parallel 818. As previously indicated, parallel processing comprises subdividing or partitioning the analysis into sub-processes that may be independently operated upon by a plurality of nodes within a clustered computer environment.

Parallelization of the data analysis commences in a state 820 where both the experimental mass analysis data and the results returned from the spectral database query 260 are split into jobs that are operated on by nodes within the cluster. In this state 820, information is extracted and stored in fields of tables which are integrated into the database schema. As shown in subsequent figures, these tables are populated with information which characterize each peptide component and provide links or associations to allow the information stored in the tables to be analyzed and correlated.

In a subsequent state 825 the information retrieval module 210 of the data analysis system 210 may additionally acquire supplemental information from other external or bioinformatic databases 254 which is desirably associated with the experimental results and peptide-correlated output file information. This supplemental information may, for example, include descriptions and information further detailing the matched peptides from FASTA databases, as well as, other sources of information such as GenBank search results and nucleic acid expression data.

Additional information may be computed by the data analysis system 200 in a state 830 where parameter calculations based on the associated data are made. In this state 830, the information contained in the fields of the tables may be used to calculate information such as the molecular weight of the peptides undergoing analysis, charge distributions, or other information which may be of interest to the investigators. Furthermore, links or associations may be created within the tables which serve as pointers or hyperlinks to the stored mass spectra or peptide-correlated output files 260 to facilitate subsequent investigator retrieval of the information stored in the database 226.

As each node completes the aforementioned operations to prepare and analyze the subset of information which has been distributed to it, the process enters a state 835 where the information is uploaded to the database 226. This state 835 utilizes the database 226 as a centralized storage area to organize the data output 208, peptide-correlated output files 260, and any newly created information/associations in a manner that is readily accessible to the investigator. Additionally, the informational upload 835 to the database 226 prepares the data analysis system 200 for subsequent operations in which differential analysis and proteomic expression evaluation are performed. The process 800 subsequently reaches an end state 842 where the informational processing and upload is complete and the data analysis system 200 made ready to perform other functions.

The foregoing method of parallel data processing efficiently acquires the necessary data and information to associate the experimentally obtained mass spectra with spectra obtained from known peptide sequences. This method may further be scaled up or down as necessary to accommodate various amounts of data and provides an improved method for populating the bioinformatic database 227 so as reduce the amount of time necessary to complete the analysis of the experimental results.

A distinctive feature of the data analysis system 200 resides in its ability to dynamically create links or identifiers during the processing of the data output 208 and sequence-correlated data output files 260. These links are automatically created and stored in the bioinformatic database 227 in response to a number of definable events which the data analysis system 200 is programmed to recognize. In one aspect, when a particular database match or sequence homology is encountered with a peptide undergoing analysis. The data analysis system 200 may create the identifier which flags the data of interest for subsequent review by the investigator.

The identifier may additionally comprise a hyperlink to an actual image of the spectrum stored in the database 227 whereby the investigator can quickly review the visual representation (picture) of the mass analysis. These identifiers are desirably stored in the database 227 and may be subsequently used by the investigator to selectively retrieve data of interest. Additionally, the investigator may create similar links or identifiers in a user-defined manner to flag desired data or information selectively.

The hyperlinked association of data and information can also be represented by a link which contains the address of a computer that runs script to generate an image of the spectrum on the fly, based upon the numerical values of the mass spectrum analysis. Thus, actual images of the spectrum need not necessarily be stored in the database 227 and may instead be generated upon request of the investigator.

In one embodiment, images of the experimental spectrum are desirably stored within the database to provide an additional source of information which may be used for data analysis. For example, neural network analysis of the images of the experimental spectrum may be performed to aid in the identification of proteomic differences and data mining operations. In a neural network processing paradigm, information is analyzed by methods such as pattern recognition or data classification. Furthermore, the neural network is an adaptive process that “learns” or creates associations based on previously encountered data input. The storage of images within the database 227 therefore may be desirably used in conjunction with the neural network processing paradigm to provide improved information analysis as compared to using more traditional processing methodologies alone. Furthermore, storage of images within the database 227 improves access times for investigators wishing to view the mass spectrum compared to that of rendering the images from the numerical representations of the data and information.

As described below with relation to Table 6, the system also provides a convenient means for organizing and querying the data files that are returned from the spectral database. A queryable filesystem (QFS) brings together the ease of data storing of a regular file system with the ease of complex querying associated with a relational database. The actual content of this file storage device is delivered by a number of daemons running on one or more servers within the data analysis system. In one embodiment, the daemons are: an NFS daemon, which makes the files available to UNIX machines; a SAMBA daemon, which makes the files available to Windows machines and a database server (MySQL, PostgreSQL or Oracle)—which makes the SQL tables available to any client. For platform independency, these tables can be delivered using the ODBC protocol or, for improved access speed, a specific database connection can be made.

The contents of the real file system and of the relational database are kept in-sync by the client that browses their contents. When a file is deleted or a table is dropped, the appropriate changes are made in the description tables. If, for any reason, the QFS browser does not perform this task, a periodically scheduled task will check the consistency of the description tables to make sure that their content is accurate. Entries that correspond to non-existing data are deleted and data that does not have an entry in the description table is moved to a folder called “Orphans”. The system logs these changes such that appropriate actions can be taken by the operator.

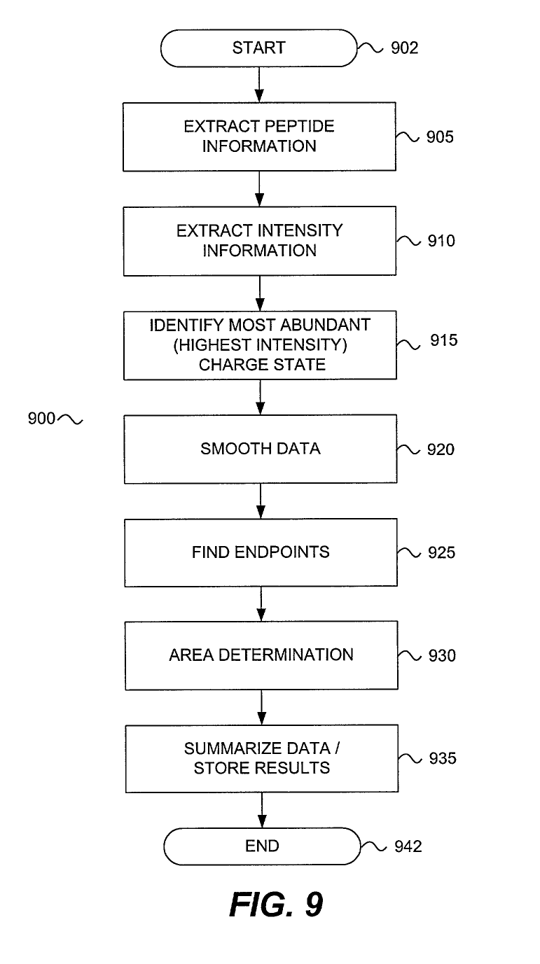

FIG. 9 provides a detailed flow diagram of a quantification method 900 used by each node during parallel peptide assessment. Beginning in a start state 902 the process advances to a state 905 where quantification is performed by extracting peptide information from the relevant correlated database files 260 and comparing this information with the peptide associated peak or intensity curve 407 undergoing analysis. One component of the correlated database file 260 comprises a summary of expected peaks and intensities at various charge states for the associated known peptide sequence. These peaks and intensities are extracted in a subsequent state 910 to within one atomic mass unit (amu) of the calculated masses of the peptide at the different charge states which the peptide exists as during the mass analysis. During this stage 910, appropriate peaks are isolated from the spectrum to isolate and identify relevant portions of the spectrum from which quantitation will subsequently be made.

As will be appreciated by those of skill in the art, during mass analysis, peptides resolved in the primary mass spectrum are present in a number of different charge states. These charge states are indicative of states of ionization of the peptide when subjected to the energy of the mass analysis. Each ionization state results in a different mass-to-charge ratio for the peptide and results in a plurality of independently resolved peaks or charge intensities appearing in the primary spectrum. The exact number of peaks or charge intensities is therefore dependent on the number of different charges states possible for each peptide.

One feature of the quantification method 900 resides in its ability to identify the aforementioned charge states for each peptide and determine which charge states are appropriate for assessing quantitation. To accomplish this task, the quantification method 900 enters a state 915 to determine the most abundant charge state of the peptide undergoing analysis based on the expected charge states for the associated known peptide. In one embodiment, the most abundant charge state is identified by extracting stored peptide intensities from the correlated database file 260 to identify peaks in the mass spectrum which correlate with the plurality of charge states of the peptide under analysis. During this state 915, the node identifies the highest intensity charge state and takes the peak 146 associated with this charge state to be the most relevant for the purposes of quantitation.

Upon identifying the peak 146 of the mass spectrum to be quantified, the quantification method 900 proceeds to a state 920 where a numerical filter is used to smooth the data contained in the identified peak 146 of the mass spectrum. In one aspect the numerical filter comprises a Butterworth or Chebyshev filter applied to the peaks 146 of the mass spectrum to isolate each peak of interest from any intervening peaks or background noise. Subsequently, the method proceeds to a new state 925 wherein an endpoint determination is made to define the bounds of the peak area to be quantified. The peak smoothing and endpoint identification states 920, 925 are useful in isolating the peptide-associated peak of interest, for which quantitation of peak area will be made, from any background noise or other closely positioned peaks within the mass spectrum.

The method 900 then proceeds to a state 930 where an area determination 930 is made to determine the relative amount of peptide present. Information related to the calculated peak area and quantitation of the peptide is subsequently summarized to a file or table in a new state 935 and is written back to the working database 226 for storage in the bioinformatic database 227.

In another embodiment, the method 900 contains modules for optimizing the peptide data stored in the correlated database file 620. A duplicate peptide module will detect identical peptides (with the same marker or label) that have been identified in immediately adjacent peaks. This result may be due to a long elution time for a particular peptide, so that the measured peak for the peptide extends beyond the dynamic exclusion window specified by the researcher. Thus, the area beyond the exclusion window is detected as a separate, second peak. By comparing the back border value of the first peak with the front border value of the second, the module may detect that the second peak is in fact the tail end of the first. In that case, the module will combine their areas and record that value as the area of the first peak while eliminating the second peak from the data set. A sanity check module eliminates duplications in Sequest peptide identity files due to multiple matches with different cross correlation (Xcorr) scores for a single detected peptide. The module will select Sequest files with identical identity matches. If the files originate from peptides with the same step and charge states, the module will compare the Sequest files for that peptide and select the Sequest file with the highest cross correlation value for entry into the correlated database file 620. In another embodiment, the module will also eliminate duplication in Sequest identity file matches due to multiple charge states for a particular peptide, selecting the peptide with the plus two charge state.

As will be appreciated by those with skill in the art, a plus two charge state for a large ion such as a peptide will result in a m/z ratio number that falls in the optimal detection range in terms of sensitivity and precision for some types of detectors used in mass spectroscopy.

The aforementioned quantitation method 900 defines a principle functionality of the distributed node processing for each resolved peptide 146 in the primary mass spectrum. This method 900 features an efficient peak isolation and quantitation approach that identifies the most relevant peak associated with a peptide having a plurality of charge states. Furthermore, the identified mass spectrum associated with each peptide of interest is isolated from the surrounding information contained in the spectrum so that an accurate assessment of the peak area may be obtained. This feature of the invention contributes to increased sensitivity in identifying relative peptide abundances and improves the determination of proteomic differences when comparing analogous peptides within the mass spectrum.

4. Exemplary Pseudocode for Parallel Processing

The following pseudocode illustrates one example for implementing a parallel processing routine for analysis of the primary mass spectrum and subsequent determination of peptide quantitation and proteomic differences. A master/slave paradigm is used to perform the calculations associated with the data analysis and, as previously indicated, the functions are implemented in a parallel programming language such as PVM, MPI or LSF. The comments provided within the pseudocode describe the functionality of the procedure calls used to perform the data analysis which can be coded in numerous different ways as will be appreciated by one of skill in the art.

The software of the data analysis system 200 therefore desirably provides easy and open access to data contained within the relational database 227 and is designed to be independent of system architecture. These features permit the software to be readily extended to larger scale installations to accommodate the vast quantities of data which are typically associated with identifying and comparing the many thousands of peptides found in most biological samples.

a. Pseudocode for Parallel Processing (Master)

/* start by building the parallel virtual machine - see how many nodes (slaves)
are available and what is their computational load; launch slave tasks on the
remote nodes */

initiate(parallel_virtual_machine);

/* the master node first compiles a list of all the output files from the
spectral database search; these files (*.out) contain information regarding
the matched peptides from a given database such as the correlation score, the
preliminary score, the sequence, the number of matched ions and so on */

read(*.out files);

/* once this list has been compiled, workload packets need to be constructed;
these are sublists of output files, computed such that the total number of
matches per packet is constant. This guarantees a fair workload for all the
slaves in the cluster */

compute(workload);

/* next the summary parameters are broadcasted to the slaves, e.g. - FASTA
database used for search and/or description, database to be uploaded with the
results from the search */

broadcast (parameters);

/* here the main work of the master begins: keep sending workload packets to
the nodes */

while (there is work to be done) {

wait(request from slave);
send(workload_packet, slave);
receive(acknowledgement);

}

/* when there is no more work to be done, signals are sent to the slaves in
the cluster so they can exit gracefully */

shutdown(slaves);

/* and the process is finished */

exit;

b. Pseudocode for Parallel Processing (Slave)

/* once the slave process has been started, it needs to know the general
parametes of the parallel job */

receive(broadcasted parameters);

/* signal to the master node that we are ready to begin */

| | |
|---|---|
| 1 | communicate(availability to master); |

/* meet all the communication requirements imposed by the master:

get ready to receive workload packet... */
receive(workload_packet);

/* acknowledge the transmission */

send(acknowledgement);

/* examine the workload packet; open the corresponding output files */

forall(files in workload_packet) {

open(file);

/* make connection with the database that stores the search summary */

initiate(database_connection);

/* and now start the real work: get all the details for each hit... */

forall(entries in file){

get_search_results (entry);
compute_peptide_molecular_weight (entry);
get_description_from_fasta-db(entry); /* this

| | |
|---|---|
| is | the database that Sequest used */ |

/* and upload the details */

upload_db(tablename, entry.details);

}

}

/* done with this packet of data - communicate the master that we are ready
for more */

goto (1);

D. Exemplary Data Tables for Storing Spectral Data

The following Tables illustrate a schema that may be used in the relational database 227 for storing and processing the aforementioned mass spectra. Experimental information, data output and subsequent results from spectral database queries are stored in fields of these Tables and are used in the identification of proteomic differences between the two or more biological samples. As previously described, these Tables are desirably implemented using a specialized database programming language such as SQL or MySQL in order to permit the fields and information stored in these Tables to be flexibly associated. This implementation also provides search, query, and processing routines used to identify the primary mass spectrum peaks. The information retrieved from the spectral database 250 and stored in the Tables is further used to associate peptide-specific sequences with the primary mass spectrum peaks, and assess differential peptide expression between analogous peptides in the mixed-population. It will be appreciated that the following combination of Tables illustrate one of many possible schemas that may be used to process and analyze the mass spectral data and evaluate peptide expression. As such, other implementations and Table schemas should be considered to be but other embodiments of the present invention.

Tables 1 and 2 illustrate peptide and peptide tables or entities that store information about the peptides and peptides identified by mass spectral analysis. In these tables, the peptide and peptide entities are defined by a plurality of fields which identify features and information related to the peptide. The peptide and peptide entities, as well as other related entities, serve as a basis for storing and associating information useful in identifying the peptides, relating the peptides with the mass spectra information, and describing information that may be of interest to the investigator.

Each field may additionally be associated with a number of database properties or attributes used to define the type of data in the table and describe functionality used by the relational database to manipulate the information within the table. For example, each field of the table may be associated with attributes including: Type, Null, Key, Default, and Extra. The Type attribute defines the type of information or value which is to be stored within the table such as an integer, character, text, or other variable identifier. The Null attribute indicates whether the field must contain an associated data value or may be stored within the relational database as an empty field. The Key attribute defines a unique instance of the entity and is used by the relational database 227 to maintain links or associations in the table and interrelate the table with other tables in the database 226. The Default attribute defines the contents of the field when an instance of the Table is created in the database 226, 227. The Extra attribute defines properties or functionality which the database programming language uses to perform operations on fields of the table such as auto incrementing values to facilitate user interaction.

Table 1 further comprises a peptide_id field (defines a unique peptide identifier for the matched peptide), a name field (defines the name of the peptide), and a sequence field (defines the peptide sequence). These fields define attributes of the Peptide entity which may be associated with other fields of other tables or entities to aid in the organization of the database schema. In a similar manner, Table 2 comprises a peptide_id field (defines the unique peptide identifier for the matched peptide), a name field (defines the name of the peptide sequence, with the corresponding peptide belonging to the named peptide), and a peptide_id field (defines a unique peptide identifier for the corresponding peptide).

Table 3 illustrates a global table that is used in conjunction with peptide and peptide tables to store and relate information used in the processing of the tandem mass spectra obtained from the spectral database 250. The fields of this table comprise: a peptide_id field (defines a peptide identifier similar to that of the peptide and peptide tables), a species field (defines species, conditions, or treatments of the biological samples), a charge_state field (defines the charge state of the peptide of interest), a quantitation_value field (defines the computed quantitation value), a ratio field (defines the relative abundance of one biological sample to another), a mass field (defines the mass of the peptide), a identified_charge_state field (defines the charge state of the peptide as identified by the spectral database or the data analysis program 200), and a duplicate field (defines whether or not the peptide has been found elsewhere in the mass spectrum or database).

Table 4 illustrates a quantitation table used by the data analysis program 200 to maintain state information and run indicators used in the identification and quantitation of the peaks of the primary mass spectrum. The fields of this table comprise: a run_id field (defines the identifiers used by the data analysis program 200 to determine what operations are being performed), a Qvalue field (defines the quantitation value obtained by the data analysis program), a start_scan field (defines a number corresponding to the scan number where the peak under analysis starts), end_scan (defines a number corresponding to the scan number where the peak under analysis ends), a duplicate field (defines whether or not the peptide is a duplicate), a xcorr field (defines a correlation score as computed by the spectral database analysis), a DCn field (defines a delta Cn value as computed by the spectral database analysis), a valley field (defines whether or not the start_scan analysis commences in a valley of the spectrum), and an extrapolation field (defines whether or not extrapolation has been performed during the analysis).

Table 5 illustrates a node table used by the data analysis system 200 as a data structure to pass information between nodes of the parallel computing distributed system for data analysis. The fields of this table comprise: a dirname field (defines a name of a directory which contains the data files 260 produced by the spectral database 250), a filename field (defines the filenames of the data files 260 files produced by the spectral database 250 and may include a hyperlink to the actual raw spectrum data), a charge state field (defines the charge state [1,2 or 3] for the top rated peptide in a given data file 260), a mass field (defines the mass of the peptide), a tol field (defines the mass tolerance of the analysis), a tot_icurrent field (defines the total ion current per mass spectrum), a Xcorr field (defines the correlation score for the peptide), a dCn field (defines the delta Cn between the peptide and one defined in the data file 260), a Sp field (defines a preliminary scoring of the peptide under analysis), a RSp field (defines a ranking for the preliminary scoring of the peptide under analysis), a IonsMatch field (defines the number of matched ions found in the mass spectrum), a IonsTot field (defines the total number of ions expected), a SpecLink field (defines a hyperlink to a plot of the actual spectrum), a PeptideWeight field (defines the weight of the peptide under study), a resultPI field (defines the pH of the peptide at the specified temperature), a Ref field (defines a database reference for the matched peptide), a DuplicateCount field (defines a number of places where the peptide occurs and may further contain a hyperlink to other information such as BLAST sequence information), a tryptic field (defines the tryptic nature of the peptide), a Sequence field (defines the actual sequence of the peptide under study), and a PeptideHeader field (defines references and annotations for the matched peptide).

Table 6 provides a means for being able to provide the user with the ability to browse the data stored in relational tables just like they would browse the files on their computer using some file manager (e.g., Windows Explorer). Individual tables with unique names (“uniquename”) are created to store the data from each time that a differential analysis is run through the system. Table 6 is updated as to reflect the creation of a new data table from the data located in a certain directory (the field “PWD” stores the name of this directory). The fields “oldname” and “olddb” are pseudo-names associated with a given table—names that will be visible to the user. The field “useradded” is a flag that describes whether or not the table has been created by an actual experiment, or is from the result from a data query. The field “rows” is the number of rows in the table. The field “createtime” includes the date and time when the table was created and the field “description” provides a user-supplied description of the table, to be associated with the data stored in the table. Finally, the field “creator” is the username of the person who ran the gathered the data and provided the description.

A number of health checks are performed by the system on Table 6. For example, if a unique table associated with the data currently searched already exists, the user has the options of deleting the existing table or continuing to upload data into the existing table. Of course, a first check is made to determine if this unique table indeed exists or the table is corrupted.

Table 6 can be used as part of a special-purpose application that allows easy and intuitive access to database results. A file manager performs repeated queries to retrieve the names of the (unique) tables associated with the location of the current directory (by means of querying on the PWD variable). Thus, Table 6 provides the software hooks for a virtual file system on top of an existing relational database, one that would mirror the hierarchy of a real file system—the one holding raw data and raw data analysis results.

The aforementioned tables and descriptors summarize some of the primary fields and attributes associated with performing the data analysis used to identify the sequence of each peak within the primary mass spectrum. Furthermore, these tables are used by the data analysis system 200 to store the information useful in comparing the analogous peptides in the mixed-population and to identify proteomic differences using the data analysis system peak identification algorithms.

TABLE 1
PEPTIDE

| Field | Type | Null | Key | Default | Extra |
|---|---|---|---|---|---|
| peptide_id | int (11) | | PRI | NULL | auto_increment |
| name | varchar (255) | YES | | NULL | |
| sequence | mediumtext | YES | | NULL | |

TABLE 2
PEPTIDE

| Field | Type | Null | Key | Default | Extra |
|---|---|---|---|---|---|
| peptide_id | int (11) | | | 0 | |
| sequence | varchar (255) | YES | | NULL | |
| peptide_id | int (11) | | PRI | NULL | auto_increment |

TABLE 3
GLOBAL

| Field | Type | Null | Key | Default | Extra |
|---|---|---|---|---|---|
| peptide_id | int (11) | | | 0 | |
| species | tinyint (4) | YES | | NULL | |
| charge_state | tinyint (4) | YES | | NULL | |
| quantitation_value | float | YES | | NULL | |
| ratio | float | YES | | NULL | |
| mass | float | YES | | NULL | |
| identified_charge_state | tinyint (4) | YES | | NULL | |
| duplicate | tinyint (4) | YES | | NULL | |

TABLE 4
QUANTITATION

| Field | Type | Null | Key | Default | Extra |
|---|---|---|---|---|---|
| run_id | tinyint (4) | YES | | NULL | |
| qvalue | float | YES | | NULL | |
| start_scan | smallint (6) | YES | | NULL | |
| end_scan | smallint (6) | YES | | NULL | |
| duplicate | tinyint (4) | YES | | NULL | |
| XCorr | float | YES | | NULL | |
| DCn | float | YES | | NULL | |
| valley | tinyint (4) | YES | | NULL | |
| extrapolation | tinyint (4) | YES | | NULL | |

TABLE 5
NODE

| Field | Type | Null | Key | Default | Extra |
|---|---|---|---|---|---|
| dirname | varchar (255) | YES | | NULL | |
| filename | varchar (255) | YES | | NULL | |
| charge_state | tinyint (4) | YES | | NULL | |
| Mass | float | YES | | NULL | |
| tol | float | YES | | NULL | |
| tot_icurrent | float | YES | | NULL | |
| XCorr | float | YES | | NULL | |
| dCn | float | YES | | NULL | |
| Sp | float | YES | | NULL | |
| RSp | smallint (6) | YES | | NULL | |
| IonsMatch | smallint (6) | YES | | NULL | |
| IonsTot | smallint (6) | YES | | NULL | |
| SpecLink | varchar (255) | YES | | NULL | |
| PeptideWeight | mediumint (9) | YES | | NULL | |
| resultPI | float | YES | | NULL | |
| Ref | text | YES | | NULL | |
| DuplicateCount | varchar (255) | YES | | NULL | |
| tryptic | tinyint (4) | YES | | NULL | |
| Sequence | text | YES | | NULL | |
| PeptideHeader | text | YES | | NULL | |

TABLE 6
EXPLORING

| Field | Type | Null | Key | Default | Extra |
|---|---|---|---|---|---|
| PWD | text | YES | | NULL | |
| uniquename | int (10) unsigned | | PRI | NULL | auto_increment |
| oldname | varchar (64) | YES | | NULL | |
| olddb | varchar (64) | YES | | NULL | |
| useradded | int (10) unsigned | YES | | NULL | |
| rows | int (10) unsigned | | | 0 | |
| createtime | datetime | YES | | NULL | |
| description | text | YES | | NULL | |
| creator | varchar (100) | YES | | NULL | |

E. Peptide Labeling Methods

Embodiments of this invention provide analytical reagents and mass spectrometry-based methods using these reagents for the rapid and quantitative analysis of proteins or protein function in mixtures of proteins. The analytical method can be used for qualitative and particularly for quantitative analysis of global protein expression profiles in cells and tissues, i.e., the quantitative analysis of proteomes. The method can also be employed to screen for and identify proteins whose expression level in cells, tissue or biological fluids is affected by a stimulus (e.g., administration of a drug or contact with a potentially toxic material), by a change in environment (e.g., nutrient level, temperature, passage of time) or by a change in condition or cell state (e.g., disease state, malignancy, site-directed mutation, gene knockouts) of the cell, tissue or organism from which the sample originated. The proteins identified in such a screen can function as markers for the changed state. For example, comparisons of protein expression profiles of normal and malignant cells can result in the identification of proteins whose presence or absence is characteristic and diagnostic of the malignancy.

In an exemplary embodiment, the methods herein can be employed to screen for changes in the expression or state of enzymatic activity of specific proteins. These changes may be induced by a variety of chemicals, including pharmaceutical agonists or antagonists, or potentially harmful or toxic materials. The knowledge of such changes may be useful for diagnosing enzyme-based diseases and for investigating complex regulatory networks in cells.

The methods herein can also be used to implement a variety of clinical and diagnostic analyses to detect the presence, absence, deficiency or excess of a given protein or protein function in a biological fluid (e.g., blood), or in cells or tissue. The method is particularly useful in the analysis of complex mixtures of proteins, i.e., those containing 5 or more distinct proteins or protein functions.

One method employs affinity-labeled protein reactive reagents that allow for the selective isolation of peptide fragments or the products of reaction with a given protein (e.g., products of enzymatic reaction) from complex mixtures. The isolated peptide fragments or reaction products are characteristic of the presence of a protein or the presence of a protein function, e.g., an enzymatic activity, respectively, in those mixtures. Isolated peptides or reaction products are characterized by mass spectrometric (MS) techniques. In particular, the sequence of isolated peptides can be determined using tandem MS (MS)ⁿ techniques, and by application of sequence database searching techniques, the protein from which the sequenced peptide originated can be identified.

I. Peptide Labeling Reagents

Embodiments of the present invention provide trifunctional synthetic reagents that can be used for reducing the complexity of peptide mixtures by labeling peptides at a specific amino acid residue and then selectively enriching only those peptides containing the labeled amino acid. By preparing this reagent in two forms with detectably different masses, this technique can be used to provide accurate relative quantification of peptide amounts using mass spectrometry.

In some embodiments of the invention, the peptide labeling moiety consists of a lysine residue modified with an iodoacetamide functional group on the ε-amino group of the side chain. The synthetic peptides contain two additional motifs: a peptide epitope tag for high affinity purification; and a highly specific protease site for releasing the affinity purified labeled peptides from the affinity matrix. In addition, these synthetic peptides can readily be prepared as isoforms of two different masses by the simple expedient of using an ornithine in place of lysine to introduce a 14 mass unit difference in the carboxyl terminal acid.

In other embodiments of the invention, the peptide labeling moiety consists of a molecule modified with an iodo-containing organic substituent, which may be an iodide on a primary carbon, an acid iodide, or an iodoacetamide functional group. In addition, the peptide labeling moiety comprises a substituted benzyl moiety, which undergoes heterolytic cleavage upon exposure to light of a certain wavelength. In addition, these molecules can readily be prepared as isoforms of two different masses by the simple expedient of using an alkylene chain that has additional methylene groups or is missing methylene groups to introduce an integer multiple of 14 mass unit difference in the carboxyl terminal acid.

F. Conclusion

Briefly summarizing one embodiment of the present invention, upon receiving the results of the quantitation of the resolved peptides 146, the data analysis system 200 compares the relative peptide expression levels for the analogous peptides with different markers 122, 124. Using the quantitation module 230, the system 200 then identifies each recognizable peak or intensity curve 407 and associates any differentially tagged partner peptides (analogs). These tagged partner peptides can be recognized as peaks or intensity curves 407 that are present at a predicted mass displacement distance, based on the mass differential created by the markers 122, 124. If a potential partner peak or intensity curve 407 is found, the peptide-correlated output files 260 may be used to confirm or deny the sequences of the peptides to establish if peptides being compared are partners. This process is repeated until all possible pairs of peptide partners have been identified in the data set. The data processing module 225 then integrates the area contained by each peak or intensity curve 407 and calculates the ratio of the quantitated peaks to identify differences in peptide expression.

In a subsequent analysis stage, the data output comprising the identified differences in peptide expression can be sorted and presented to the investigator in the form of one or more reports. These reports may be categorized by identification of the peptide constituents of the mixed-peptide population, ratios of peptides containing different markers 122, 124, names of the peptides identified by the data analysis system 200, or other user-defined criteria. Additionally, the identification reports may list any unpaired peaks in the mass spectrum ordered by confidence level, peptide name, or other user-defined criteria.

The data analysis system 200 and related methods feature a significantly improved means of identifying proteomic differences between two or more biological samples. The use of markers 122, 124 with similar chemical and physical properties further serves as a basis for selective identification of peptides originating from each biological sample and permits the samples to be mixed for simultaneous mass analysis. Analysis in this manner not only improves the throughput of identification but also provides an ideal mutual internal standard for quantification which helps to increase identification accuracy and sensitivity.

Although the foregoing description of the invention has shown, described and pointed out novel features of the invention, it will be understood that various omissions, substitutions, and changes in the form of the detail of the apparatus as illustrated, as well as the uses thereof, may be made by those skilled in the art without departing from the spirit of the present invention. Consequently the scope of the invention should not be limited to the foregoing discussion but should be defined by the appended claims.

What is claimed is:

1. A method for storing peptide data to a database, comprising:
acquiring spectral maps for one or more of the peptides within the peptide mixture;
comparing the spectral maps for the one or more peptides with a first database comprising spectral maps and peptide identifications of known peptide sequences;
receiving a plurality of output files from the first database, wherein the output files comprise information which identify associations between the one or more of the peptides in the peptides mixture with known peptide sequences in the first database;
parsing the output files to organize and associate the information contained within each output file; and
storing the information in a second database.

2. The method for storing peptide data of claim 1, wherein the spectral maps are acquired by performing a first mass analysis to resolve the plurality of peptides in the peptide mixture.

3. The method for storing peptide data of claim 2, wherein the spectral maps are further acquired by performing a second mass analysis of each of the resolved peptides to produce the spectral maps characteristic of the sequence of the resolved peptides.

4. The method for storing peptide data of claim 3, wherein the first and the second mass analysis comprise mass analytical techniques selected from the group consisting of: electron ionization mass analysis, fast atom/ion bombardment mass analysis, matrix-assisted laser desorption/ionization mass analysis and electrospray ionization mass analysis.

5. The method for storing peptide data of claim 1, wherein the spectral maps are compared with the first database by a data analysis program which identifies similarities in features of the spectral maps to associate the spectral maps of each of the peptides in the peptide mixture with the spectral maps of the known peptide sequences.

6. The method for storing peptide data of claim 5, wherein the identified similarities in features of the spectral maps are used by the data analysis program to identify the peptide sequences for the peptides in the peptide mixture.

7. The method for storing peptide data of claim 6, wherein the output file comprises a summary of the identified similarities in features of the spectral maps of the peptide mixture and the peptides of known sequence stored in the first database.

8. The method for storing peptide data of claim 1, wherein the spectral maps are stored to the second database in addition to the peptide data.

9. A data analysis system for storing peptide data, comprising:
a first module configured to receive peptide mass spectral data from a mixture of peptides;
a second module configured to compare the peptide mass spectral data with a first database of stored mass spectrum and retrieve a plurality of textual analysis files, wherein each text file provides an identification of a protein corresponding to a peptide in the mixture of peptides;
a third module configured to receive the textural analysis files from the first database and store the information from the textural analysis filed into a second database.

10. The data analysis system of claim 9, wherein the mixture of peptides is a mixture of labeled peptides.

11. The data analysis system of claim 10, wherein the mixture of peptides comprises a first group of peptides that are labeled with a first label, and a second group of peptides that are labeled with a second label.

12. The data analysis system of claim 11, wherein the first label has a first mass and the second label has a second mass.

13. The data analysis system of claim 12, comprising a fourth module that determines the relative abundance of a peptide in the first group of peptides with the abundance of the same peptide in the second group of peptides.

14. The data analysis system of claim 9, wherein the second database is also configured to store a copy of the peptide mass spectral data corresponding to each textural analysis file.

15. The data analysis system of claim 9, wherein the second database is configured as a server and can be accessed through a browser software program.

16. The data analysis system of claim 9, comprising a first table that stores a unique identifier of each textural analysis file.

17. The data analysis system of claim 16, comprising a file query program that displays the name of each textural analysis file by reference to its unique identifier in the first table.

18. The data analysis system of claim 9, wherein the peptide mass spectral data tandem mass spectra from a tandem mass spectrometer.

19. The data analysis system of claim 9, further comprising an interface to a third database of peptide information for retrieving additional information on each identified protein.
